SEQUENCE LISTING (13 sequences)

SEQ ID NO: 1 - DNA, 811 bp, Artificial Sequence
  Nucleotide sequence containing the codon optimized taqI gene
gctagccacaaaaatggcttccactcaggctcagaaggctcttgagactttcgagcgatttctcgcttctcttgacctggagtcctaccagcagaagtaccgacccatcaagactgttgagcaggaccttccccgagagctgaacccccttcccgacctgtacgagcactactggaaggcccttgaggataacccctccttcctgggcttcgaggagttctttgaccactggtgggagaagcgacttcgacccctcgacgagttcattcgaaagtacttttggggttgctcctacgcctttgttcgacttggccttgaggctcgactgtaccgaactgccgtttccatctggactcagtttcacttctgctaccgatggaacgcctcctgcgagcttccccttgaggccgcccccgagcttgacgcccagggcattgacgccctgattcacacttccggttcctctaccggaatccagatcaagaaggagacttaccgatccgaggccaagtccgagaaccgatttctccgaaagcagcgaggcaccgccctcatcgagattccctacacccttcagactcccgaggagcttgaggagaaggccaagcgagcccgagttaacggagagacttaccgactttgggccaaggttgctcaccaccttgaccgacttgagaacggttttgtcatttttcgagagtcttacgttaagtctattgagcttttcctccagaagaacgctcccaccctttctggactcattcgatgggaccgagttgctcaggaggccctcaccgccccctaaacgcgt

SEQ ID NO: 2 - DNA, 792 bp, Thermus aquaticus
  taqI gene
atggcttccacacaagcccagaaagcgctcgaaacttttgagcgttttctcgcaagcttggacctcgagtcctaccagcaaaagtaccgccctatcaaaacggttgaacaagacctgcctagggagctgaacccgcttccggacctgtacgagcattattggaaagcgcttgaggataacccttccttcctgggcttcgaagagttctttgaccactggtgggaaaagcgcctacggcccttggacgagttcatacgcaaatacttttggggatgctcctacgcgtttgttcgcttgggcctcgaggctaggctgtaccgaacagccgtttccatctggactcagtttcacttctgctaccgctggaacgcctcctgcgagcttcctctagaagctgccccagaactcgacgcccaagggatagacgcgctgattcatacaagcgggtcctcaacaggaatccagatcaaaaaggaaacttaccgttccgaggccaagagcgagaaccgctttttaaggaagcaaagaggcaccgccctcatcgagattccctacaccctgcagacaccagaggagctcgaagaaaaagccaaacgggcaagagtgaacggagaaacctaccgtctatgggccaaggttgcacaccatttggaccgtctagaaaacggattcgtcatttttcgggaaagttatgtgaaaagcattgagctttttctccagaaaaacgctcctaccctatctgggctcatccgctgggacagggtggcccaggaagccctcaccgccccgtga

SEQ ID NO: 3 - protein, 263 aa, Thermus aquaticus
  Amino Acid sequence of TaqI nuclease
MASTQAQKALETFERFLASLDLESYQQKYRPIKTVEQDLPRELNPLPDLYEHYWKALEDNPSFLGFEEFFDHWWEKRLRPLDEFIRKYFWGCSYAFVRLGLEARLYRTAVSIWTQFHFCYRWNASCELPLEAAPELDAQGIDALIHTSGSSTGIQIKKETYRSEAKSENRFLRKQRGTALIEIPYTLQTPEELEEKAKRARVNGETYRLWAKVAHHLDRLENGFVIFRESYVKSIELFLQKNAPTLSGLIRWDRVAQEALTAP

SEQ ID NO: 4 - DNA, 1740 bp, Yarrowia lipolytica
  carB
atgtccaagaaacacattgtcattatcggtgctggcgtgggtggcacggctacagctgctcgtttggcccgcgaaggcttcaaggtcactgtggtggagaaaaacgactttggtggcggccgctgctccttgatccatcaccagggccatcgctttgatcagggcccgtcgctctacctgatgcccaagtactttgaggacgcctttgccgatctggacgagcgcattcaagaccacctggagctgctgcgatgcgacaacaactacaaggtgcactttgacgacggtgagtcgatccagctgtcgtctgacttgacacgcatgaaggctgaattggaccgcgtggagggcccccttggttttggccgattcctggatttcatgaaagagacacacatccactacgaaagcggcaccctgattgcgctcaagaagaatttcgaatccatctgggacctgattcgcatcaagtacgctccagagatctttcgcttgcacctgtttggcaagatctacgaccgcgcttccaagtacttcaagaccaagaagatgcgcatggcattcacgtttcagaccatgtatatgggcatgtcgccctacgatgcgcctgctgtctacagcctgttgcagtacaccgagttcgctgaaggcatctggtatccccgtggcggcttcaacatggtggttcagaagctagaggcgattgcaaagcaaaagtacgatgccgagtttatctacaatgcgcctgttgccaagattaacaccgatgatgccaccaaacaagtgacaggtgtaaccttggaaaatggccacatcatcgatgccgatgcggttgtgtgtaacgcagatctggtctatgcttatcacaatctgttgcctccctgccgatggacgcaaaacacactggcttccaagaaattgacgtcttcttccatttccttctactggtccatgtccaccaaggtgcctcaattggacgtgcacaacatctttttggccgaggcttatcaggagagctttgacgaaatcttcaaggactttggcctgccttctgaagcctccttctacgtcaatgtgccctctcgcatcgatccttctgctgctcccgacggcaaggactctgtcattgtcttggtgcctattggtcatatgaagagcaagacgggcgatgcttccaccgagaactacccggccatggtggacaaggcacgcaagatggtgctggctgtgattgagcgtcgtctgggcatgtcgaatttcgccgacttgattgagcatgagcaagtcaatgatcccgctgtatggcagagcaagttcaatctgtggagaggctcaattctgggtttgtctcatgatgtgcttcaggtgctgtggttccgtcccagcacaaaggattctaccggtcgttatgataacctattctttgtgggtgcaagcacgcatcccggaactggtgttcccattgtccttgcaggaagcaagctcacctctgaccaagttgtcaagagctttggaaagacgcccaagccaagaaagatcgagatggagaacacgcaagcacctttggaggagcctgatgctgaatcgacattccctgtgtggttctggttgcgcgctgccttttgggtcatgtttatgttcttttacttcttccctcaatccaatggccaaacgcccgcatcttttatcaataatttgttacctgaagtattccgcgttcataactctaatgtcatttaa

SEQ ID NO: 5 - DNA, 19 bp, Artificial Sequence
  Primer M04641
caatctgttgcctccctgc

SEQ ID NO: 6 - DNA, 19 bp, Artificial Sequence
  Primer M04642
atcctttgtgctgggacgg

SEQ ID NO: 7 - DNA, 558 bp, Yarrowia lipolytica
  crtZ-Dc
atgcttgcgttgtggaataccgggatcgtgctactgactatcatcatcatggaaggggtggcaacgttcgcacacaagtacatcatgcacggctggggatggggctggcatcattcgcaccataccccgcgcaaaggggcgtttgagcgtaacgatctctatgcggtggtgtttgcgctactggccattgcgctgatttacgcgggcagcgaagggtactggccgcttcagtggattggcgcgggaatgaccggctacggcgtgatctactttatcgttcacgatggtcttgtccaccagcgctggccgttccgttacgtgccgcgccgcggctatctgcgccgcctctacatggcacaccggctgcatcacgcggtgcgggggcgcgaagggtgcgtctccttcgggtttatctacgccccaccggtggacaagctgcaggcggtgctgcgcgaacgtaacggcagacccgccagcgcgggcgctgccagaggtgcggatcgcgcggcggccagctcgccttccgggaagccatcgcctgcttcgcgccggaaataa

SEQ ID NO: 8 - DNA, 977 bp, Artificial Sequence
  Nucleotide sequence containing the codon optimized phoI gene
tgctagccacaaaaatggagatgtacaaggttggtcgatacctcgttgattccctccagatttacttccccgcttctcttgagatccaggaggagcttattaacaacggcttctacgttccccgatctcctgatcgaaaggtttctatgcccattcccattgtttactccgattttggtggtcgagttatttctattgagcgactcattcctcccgagtggcttgagatttctcccgagcagctcggttgggaggagacttaccttgagaacaagcgaggtttcaagctccctaaggaggaggtttacgttgatgtttctatttctaacgattccattattttcgagcttgacgttaagaactaccaccttgagcgaacctccattcgaggcatcaaccctgagaagtggaagaactgggttatgttttacattgatctcaagtacgttgatgagttcattaacgctcttcgagagcacatccctgctttcgagaacaagacccgagttattcgagagaagcagcagggtggcaaggaggttacttactacgctaaggttaacgtcaagaacttctctctttgtctcggttgtttcgatctcgctcagcgataccttcagatcaaggctaaggagcactgtaacatttaccctggttctcccacctgtaacgattctctctctaagctcaagctccgacttgagtacgatccctccattaccacctttgctaaggttggtattgccaagatttctggtaagcgaccccagatcatggttaagctcacctctaccgagactaagaccattcgaggtattctcaagcctgagattaagggtaaggcccgaggtaagctcgtttactgtgatcaccgagagaagcgacagtacattgctcttgacctcttcgatttttacaaggccctcgtttctactaagaagtacgagggtaagctccctaccgatgattaaacgcgt

SEQ ID NO: 9 - DNA, 957 bp, Pyrococcus horikoshii
  phoI gene
atggaaatgtataaggttggaagataccttgttgattcccttcaaatatattttcccgcttcactagagatccaggaggagcttataaataatggcttctacgtacccaggagcccagatagaaaagtaagcatgccaattccaatagtatactccgattttggagggagggttatatctattgagaggttaatacctccggagtggcttgaaatctcgccagagcaattaggttgggaagaaacttatttagagaacaaacgtggttttaaactacctaaagaagaggtctacgtggatgttagtataagcaacgattccataatattcgagctagacgtgaaaaattatcaccttgaaaggacatccattagaggcatcaatcctgaaaaatggaagaattgggtgatgttttatattgatttgaagtacgtagatgagttcattaacgctttaagggaacatatcccagctttcgaaaataaaacaagggttattcgtgaaaagcagcaagggggcaaggaagttacatattatgctaaagttaacgtcaagaatttcagcttatgcttaggatgcttcgatctcgctcaaaggtatcttcaaatcaaagctaaagagcactgtaacatatatccaggctcaccaacatgtaacgattctctaagcaaattaaaattaaggcttgaatacgatccatccattactacctttgctaaagttggaatagccaagatctcaggcaagagaccccagatcatggttaaattaacctctacggaaactaaaactataaggggaatacttaagccagaaataaaaggtaaggcgcgtggtaaacttgtatattgcgatcacagagaaaagagacagtatatagctttggacttgtttgatttttacaaggccttggtaagcactaagaagtatgaaggtaagctaccgactgatgattaa

SEQ ID NO: 10 - protein, 318 aa, Pyrococcus horikoshii
  Amino acid sequence of PhoI nuclease
MEMYKVGRYLVDSLQIYFPASLEIQEELINNGFYVPRSPDRKVSMPIPIVYSDFGGRVISIERLIPPEWLEISPEQLGWEETYLENKRGFKLPKEEVYVDVSISNDSIIFELDVKNYHLERTSIRGINPEKWKNWVMFYIDLKYVDEFINALREHIPAFENKTRVIREKQQGGKEVTYYAKVNVKNFSLCLGCFDLAQRYLQIKAKEHCNIYPGSPTCNDSLSKLKLRLEYDPSITTFAKVGIAKISGKRPQIMVKLTSTETKTIRGILKPEIKGKARGKLVYCDHREKRQYIALDLFDFYKALVSTKKYEGKLPTDD

SEQ ID NO: 11 - DNA, 800 bp, Thermus aquaticus
  DNA sequence encoding TaqI nuclease
ccatggcatcaacccaagcacaaaaagccctggaaaccttcgaacgcttcctggcgagcctggacctggaatcataccaacagaaataccgtccgattaaaaccgtggaacaggatctgccgcgtgaactgaacccgctgccggacctgtatgaacattactggaaagcactggaagataatccgtcctttctgggcttcgaagaatttttcgatcactggtgggaaaaacgtctgcgcccgctggacgaatttattcgcaaatatttctggggctgcagctacgcgtttgttcgtctgggtctggaagcgcgtctgtatcgcaccgccgtcagcatctggacgcaatttcatttctgctaccgctggaacgcctcttgtgaactgccgctggaagcagcaccggaactggatgcacagggcattgacgctctgatccacaccagtggcagctctacgggtatccaaatcaaaaaagaaacctaccgtagtgaagcaaaatccgaaaatcgttttctgcgcaaacagcgtggtacggctctgattgaaatcccgtataccctgcaaacgccggaagaactggaagaaaaagcaaaacgtgctcgcgtcaacggcgaaacctaccgtctgtgggcgaaagtggcccatcacctggatcgcctggaaaatggttttgtgattttccgtgaatcatacgttaaatcgatcgaactgttcctgcagaaaaacgccccgaccctgtcgggcctgattcgttgggaccgtgtcgcacaagaagcactgaccgctccgtgaggtacc

SEQ ID NO: 12 - DNA, 29 bp, Artificial Sequence
  Primer M09315
cacacaccatggcttccactcaggctcag

SEQ ID NO: 13 - DNA, 34 bp, Artificial Sequence
  Primer M09316
cacacaggtaccttagggggcggtgagggcctcc

(19) United States
(12) Patent Application Publication
(10) Pub. No.: US 2016/0186128 A1
(43) Pub. Date: Jun. 30, 2016

(54) USE OF THERMOPHILIC NUCLEASES FOR DEGRADING NUCLEIC ACIDS

(71) Applicant: DSM IP ASSETS B.V., Te Heerlen (NL)

(72) Inventors: Joshua TRUEHEART, Somerville, MA (US); Jessica Leigh, Cambridge, MA (US)

(73) Assignee: DSM IP Assets B.V., Heerlen (NL)

(21) Appl. No.: 14/772,137

(22) PCT Filed: Mar. 13, 2014
(86) PCT No.: PCT/US14/26379

§ 371 Date: Sep. 2, 2015

Related U.S. Application Data
(60) Provisional application No. 61/794,400, filed on Mar. 15, 2013.
(60) Provisional application No. 61/928,080, filed on Jan. 16, 2014.

(51) Int. Cl. C12N 1/08; C12P 23/00; C12N 9/22

(52) U.S. Cl. CPC C12N 1/08; C12N 9/22; C12P 23/00; C12Y 301/21004

(57) ABSTRACT

The present invention relates to the use of a thermophilic nuclease for degrading nucleic acids in vivo and/or in situ, wherein the thermophilic nuclease is heterologous to the host cell and is produced by the host rather than being added exogenously. The present invention further relates to a genetically modified cell which was produced according to the above method. The present invention is particularly beneficial in inactivating the biological activity of recombinant DNA in biomass or biomass-derived products.

### FIELD OF THE INVENTION

This invention relates to a method for degrading the nucleic acids of a host cell in vivo and/or in situ, in particular when the host cell comprises a recombinant DNA, using a heterologous thermophilic nuclease. The present invention is beneficial in inactivating the biological activity of recombinant DNA in a biomass. The inactivation of the biological activity of recombinant DNA helps to prevent active recombinant DNA molecules from remaining in the end product isolated from the biomass or in the biomass itself. In addition, the inactivation of the biological activity of recombinant DNA helps to prevent active recombinant DNA molecules from being released into the environment.

### BACKGROUND OF THE INVENTION

Biotechnological production processes are increasingly employed to obtain biological compounds and fine chemicals. The progress of molecular biology techniques makes possible the mass production of a wide variety of biological compounds and fine chemicals such as proteins, antibodies, polysaccharides, antibiotics, amino acids, vitamins, alcohols, etc. Often, in order to increase the efficiency of the production, the gene(s) producing the desired end product(s) is (are) genetically modified and/or introduced into a heterologous organism. The production of the desired end product(s) then takes place in fermenters controlled by modern control techniques. The end products are either the cells themselves, extracted from the cells, or collected from the cell culture broth if the compounds are found therein (either by active or passive secretion processes or cell lysis).

It is common to see that the biomass produced at the end of the fermentation process contains the desired end product(s) but also active DNA molecules. Often, the active DNA molecules are recombinant DNAs. If left untreated, the recombinant DNA molecules could remain in the isolated end product and could also be released into the environment. There have been great concerns by the general public about the possible adverse effect of the remnant recombinant DNA materials in crops and food products on human health. In addition, concerns about the potential impact of the recombinant DNA on the environment have caused the authorities and institutions in most countries to issue statutory requirements and regulations calling for inactivation of the waste materials produced from the fermentation process before being released into the environment.

There are several ways to inactivate nucleic acids. For example, nucleic acids can be inactivated physically, and most commonly, by heat. U.S. Pat. No. 5,417,862 reports a method of inactivating the biological activity of DNA by heating the DNA to 60-100° C. in the presence of an acid. A similar method of degrading DNA by a combination of heat and acid is reported in U.S. Pat. No. 5,118,603. The heating methods require considerable amounts of energy, which add to the costs of the end product in large scale fermentation processes. Furthermore, depending on the nature of the end product, the harsh heat (and/or acidic) conditions could be detrimental to the integrity and activity of said product.

The nucleic acids can also be inactivated by other physical means. U.S. Pat. No. 6,165,711 reports a method using laser beams for disintegrating nucleic acids in a biologically active proteinaceous material. As another example, a method for inactivating microorganisms using high-intensity pulsed polychromatic light is reported in U.S. patent application Ser. No. 09/818,256, now abandoned. This method requires complicated light-generating devices to be used and constantly maintained, and thus is cost-intensive. The light beams may disintegrate not only nucleic acids, but also other biological substances or active compounds which will cause them to lose their desired properties.

The nucleic acids can also be inactivated chemically by acids or alkali. For example, in U.S. Pat. No. 7,435,567, a method using hypochlorous acids for induction of autodigestion of nucleic acids in a microorganism is reported. U.S. Pat. Nos. 5,417,862 and 5,118,603 described above use other types of acid for the degradation of nucleic acids. While these methods cause the disruption of nucleic acids, the acid and alkali conditions are severe. The severe conditions may cause the unwanted denaturation of certain biological compounds such as proteins. The amount of the acid and alkali used can be relatively large, which makes the method disadvantageous from the industrial production viewpoint as well.

Attempts have been made to inactivate nucleic acids of an organism enzymatically, such as by using a nuclease. U.S. patent application Ser. No. 13/127,825 reports a method for degrading host cell nucleic acids associated with vaccine production, where the method comprises a step of nucleic acid degradation by adding purified nuclease into the cell culture. In U.S. patent application Ser. No. 10/607,903, the construction of a transgenic bacterial strain expressing a heterologous nuclease gene in an amount effective to degrade nucleic acids is reported. While the method of adding nuclease in vitro such as the one reported in U.S. patent application Ser. No. 13/127,825 causes disruption of nucleic acids, the cost is high since large amounts of nuclease are required, and the efficiency of degradation is low because the cell wall of the host cell blocks the access to nucleic acids by the nuclease when added in vitro. The transgenic approach such as the one reported in U.S. patent application Ser. No. 10/607,903 allows the nuclease to be co-expressed with the host cell and thus gain access to the host nucleic acids in vivo. However, co-expressing a nuclease in a cell without any protection mechanism, such as methylation, will significantly stress the cell, resulting in weakened cell growth and reduced production of end product.

The problem underlying the present invention is therefore to provide a cost-effective way to degrade the nucleic acids of a host cell that produces biological compounds and fine chemicals, especially on an industrial production scale. A further problem underlying the present invention is to provide a method in which the nucleic acid degradation process is controllable and does not impede the production of the desired end product(s) in the host cell. The above problems are solved according to the invention by the subject matter of the present claims.

### SUMMARY OF THE INVENTION

We have now surprisingly found that thermophilic nucleases can be used for degrading nucleic acids in vivo and/or in situ in a controlled manner. This invention relates to a novel method for degrading nucleic acids of a host organism where the host organism is modified or transformed with one or more heterologous thermophilic nuclease genes. The thermophilic nucleases disclosed in this invention are latent at temperatures at which the cell culture is normally grown to produce the desired product, but can be selectively activated at a higher temperature and thus degrade the nucleic acids of the host organism, and/or the recombinant nucleic acids integrated in the host organism, once activated. The activation can be triggered at any desired time point, such as the harvest time of the cell culture when product formation is already complete. In addition to easy control, the application of this inventive process has the benefit of acting in vivo and/or in situ and thus improving the efficiency of the enzymatic reaction and avoids the drawbacks and problems of the existing physical or chemical methods. It has thus been made possible, by application of the disclosed invention, to obtain biological compounds and fine chemicals in the form of intact cells where the active nucleic acids content of the cell, especially recombinant DNAs, is degraded and inactivated.

The practice of this invention is broadly applicable to both microorganisms and higher eukaryotic cell cultures, and particularly industrial strains used in a large scale production of commercial end products.

One aspect of the invention relates to a method for degrading the nucleic acids of a host cell in vivo and/or in situ, wherein the host cell comprises a heterologous nucleic acid sequence encoding a thermophilic nuclease gene, wherein the method comprises: a) growing the host cell at a temperature at which the thermophilic nuclease is latent; and b) degrading the nucleic acids of the host cell by changing the temperature in step (a) to a temperature at which the thermophilic nuclease is active.

The present invention also provides a genetically modified cell which comprises heterologous nucleic acid sequences encoding one or more of the thermophilic nuclease genes described above. In one embodiment, the genetically modified cell produces one or more end products, and the end products produced by the genetically modified cell are not the above thermophilic nucleases. In one embodiment, the genetically modified cell is created by introducing one or more heterologous thermophilic nuclease genes described above into a host cell. This host cell can be either a cell which has not been genetically modified, or a cell which has previously been genetically modified. This host cell, before being modified with the one or more heterologous thermophilic nuclease genes, produces one or more end products wherein said one or more end products are not the thermophilic nucleases. In another embodiment, the above mentioned host cell may be modified first with one or more thermophilic nuclease genes to create a genetically modified cell, and the genetically modified cell is subsequently genetically engineered to produce one or more other end products.

Another aspect of the invention relates to the use a thermophilic nuclease for degrading the nucleic acids of a host cell in vivo and/or in situ, wherein the thermophilic nuclease is heterologous to the host cell.

Yet another aspect of the invention relates to process for the production of a biomass product which is free of active nucleic acid molecules, wherein the biomass product is the product of a host cell comprising a heterologous nucleic acid sequence encoding a thermophilic nuclease gene, wherein the process comprises: a) fermenting the host cell at a temperature at which the thermophilic nuclease is latent; b) degrading the nucleic acids of the cell by changing the temperature in step (a) to a temperature at which the thermophilic nuclease is active; and c) recovering the biomass product, wherein the order for performing steps b) and step c) may be exchanged.

In one embodiment, the nuclease mentioned in the above aspects of invention is a DNA-degrading nuclease and the nucleic acids of the host cell are DNA. In one specific embodiment, the DNA of the host cell contains recombinant DNA. In another embodiment, the nuclease is a RNA-degrading nuclease and the nucleic acids the host cell are RNA.

In one embodiment, the temperature in step (a) of the above process invention and the method invention is optimal for the growth of the host cell. In one embodiment, the degradation is conducted at a temperature that is within about ±5° C. of the optimum temperature of said thermophilic nuclease. In a specific embodiment, the degradation is conducted at the optimum temperature of said thermophilic nuclease. In another embodiment, the degradation is conducted during the pasteurization of the host cell. In another embodiment, the degradation is conducted in a temperature range between 40° C. and 100° C.

In one embodiment, the DNA-degrading nuclease is TaqI nuclease. In one embodiment, the degradation is conducted at a temperature ranging between about 60° C. and about 70° C. In a specific embodiment, the degradation is conducted at 65° C. In one embodiment, the TaqI nuclease comprises a polypeptide having an amino acid sequence of SEQ ID NO:3. In another embodiment, the gene of TaqI nuclease comprises a nucleic acid sequence encoding a polypeptide having an amino acid sequence of SEQ ID NO:3.

In one embodiment, the DNA-degrading nuclease is PhoI nuclease. In one embodiment, the degradation is conducted at a temperature ranging between about 70° C. and about 80° C. In a specific embodiment, the degradation is conducted at 75° C. In one embodiment, the PhoI nuclease comprises a polypeptide having an amino acid sequence of SEQ ID NO:10. In another embodiment, the gene of PhoI nuclease comprises a nucleic acid sequence encoding a polypeptide having an amino acid sequence of SEQ ID NO:10.

In one embodiment, the host cell is from a plant. In another embodiment, the host cell is from an animal. In yet another embodiment, the host cell is from a microorganism. In one embodiment, the microorganism is selected from a group consisting of: yeast, fungi, algae, bacteria, and archaea. In another embodiment, the microorganism is selected from a group consisting of: Yarrowia, Bacillus, Escherichia, Pseudomonas, Paracoccus, Corynebacterium, Candida, Hansenula, Saccharomyces, Mortierella, Schizosaccharomyces, Aspergillus, Fusarium, Trichoderma, Crypthecodinium, Schizochytrium, and Thraustochytrium. In a specific embodiment, the Yarrowia species is Yarrowia lipolytica.

In one embodiment, the host cell produces one or more end products that is not the thermophilic nuclease. In one embodiment, the end product is selected from the group consisting of: phytoene, lycopene, beta-carotene, alpha-carotene, beta-cryptoxanthin, lutein, zeaxanthin, astaxanthin, canthaxanthin, echinenone, 3-hydroxyechinenone, 3′-hydroxyechinenone, adonirubin, violaxanthin, adonixanthin, ubiquinone, vitamin K, vitamin E, retinol, retinal, retinoic acid, retinol palmitate, and modified forms thereof.

In one embodiment, the gene of the thermophilic nuclease is codon optimized to match the codon usage bias of the host cell. In one embodiment, the codon optimized thermophilic nuclease gene comprises the nucleic acid sequence of SEQ ID NO:1. In another embodiment, the codon optimized thermophilic nuclease gene comprises the nucleic acid sequence of SEQ ID NO:8.

In one embodiment, more than one thermophilic nuclease gene is introduced into the host cell. In another embodiment, the recombinant DNA is optimized to include additional sites of the thermophilic nuclease. In one embodiment, the recombinant DNA is optimized to include one cleavage site every 500 or fewer nucleotides.

In one embodiment, the heterologous thermophilic nuclease genes used in the present invention do not affect the production efficiency of the end product of the host cell.

### LIST OF SEQUENCES

The nucleic acid sequences listed in the accompanying sequence listing are shown using standard letter abbreviation for nucleotide bases. Only one strand of each nucleic acid sequence is shown, but the complementary strand is understood to be included by any reference to the displayed strand. In the accompanying sequence listing:

SEQ ID NO:1 is the DNA sequence encoding TaqI nuclease from Thermus aquaticus, as optimized for expression in Yarrowia lipolytica, and includes restriction sites for cloning and a 5′ ribosomal binding site.

SEQ ID NO:1:

gctagccacaaaaatggcttccactcaggctcagaaggctcttgagact
ttcgagcgatttctcgcttctcttgacctggagtcctaccagcagaagt
accgacccatcaagactgttgagcaggaccttccccgagagctgaaccc
ccttcccgacctgtacgagcactactggaaggcccttgaggataacccc
tccttcctgggcttcgaggagttctttgaccactggtgggagaagcgac
ttcgacccctcgacgagttcattcgaaagtacttttggggttgctccta
cgcctttgttcgacttggccttgaggctcgactgtaccgaactgccgtt
tccatctggactcagtttcacttctgctaccgatggaacgcctcctgcg
agcttccccttgaggccgcccccgagcttgacgcccagggcattgacgc
cctgattcacacttccggttcctctaccggaatccagatcaagaaggag
acttaccgatccgaggccaagtccgagaaccgatttctccgaaagcagc
gaggcaccgccctcatcgagattccctacacccttcagactcccgagga
gcttgaggagaaggccaagcgagcccgagttaacggagagacttaccga
ctttgggccaaggttgctcaccaccttgaccgacttgagaacggttttg
tcatttttcgagagtcttacgttaagtctattgagcttttcctccagaa
gaacgctcccaccctttctggactcattcgatgggaccgagttgctcag
gaggccctcaccgccccctaaacgcgt

SEQ ID NO:2 is the non-optimized DNA sequence encoding TaqI nuclease, from Thermus aquaticus.

SEQ ID NO:2:

atggcttccacacaagcccagaaagcgctcgaaacttttgagcgttttct
cgcaagcttggacctcgagtcctaccagcaaaagtaccgccctatcaaaa
cggttgaacaagacctgcctagggagctgaacccgcttccggacctgtac
gagcattattggaaagcgcttgaggataacccttccttcctgggcttcga
agagttctttgaccactggtgggaaaagcgcctacggcccttggacgagt
tcatacgcaaatacttttggggatgctcctacgcgtttgttcgcttgggc
ctcgaggctaggctgtaccgaacagccgtttccatctggactcagtttca
cttctgctaccgctggaacgcctcctgcgagcttcctctagaagctgccc
cagaactcgacgcccaagggatagacgcgctgattcatacaagcgggtcc
tcaacaggaatccagatcaaaaaggaaacttaccgttccgaggccaagag
cgagaaccgctttttaaggaagcaaagaggcaccgccctcatcgagattc
cctacaccctgcagacaccagaggagctcgaagaaaaagccaaacgggca
agagtgaacggagaaacctaccgtctatgggccaaggttgcacaccattt
ggaccgtctagaaaacggattcgtcatttttcgggaaagttatgtgaaaa
gcattgagctttttctccagaaaaacgctcctaccctatctgggctcatc
cgctgggacagggtggcccaggaagccctcaccgccccgtga

SEQ ID NO:3 is the amino acid sequence of TaqI nuclease, as deduced from SEQ ID NO:1.

SEQ ID NO:3:

MASTQAQKALETFERFLASLDLESYQQKYRPIKTVEQDLPRELNPLPDLY
EHYWKALEDNPSFLGFEEFFDHWWEKRLRPLDEFIRKYFWGCSYAFVRLG
LEARLYRTAVSIWTQFHFCYRWNASCELPLEAAPELDAQGIDALIHTSGS
STGIQIKKETYRSEAKSENRFLRKQRGTALIEIPYTLQTPEELEEKAKRA
RVNGETYRLWAKVAHHLDRLENGFVIFRESYVKSIELFLQKNAPTLSGLI
RWDRVAQEALTAP

SEQ ID NO:4 is the DNA sequence of the heterologous carB open reading frame harbored by a Yarrowia lipolytica strain.

SEQ ID NO:4: carB gene

atgtccaagaaacacattgtcattatcggtgctggcgtgggtggcacgg
ctacagctgctcgtttggcccgcgaaggcttcaaggtcactgtggtgga
gaaaaacgactttggtggcggccgctgctccttgatccatcaccagggc
catcgctttgatcagggcccgtcgctctacctgatgcccaagtactttg
aggacgcctttgccgatctggacgagcgcattcaagaccacctggagct
gctgcgatgcgacaacaactacaaggtgcactttgacgacggtgagtcg
atccagctgtcgtctgacttgacacgcatgaaggctgaattggaccgcg
tggagggcccccttggttttggccgattcctggatttcatgaaagagac
acacatccactacgaaagcggcaccctgattgcgctcaagaagaatttc
gaatccatctgggacctgattcgcatcaagtacgctccagagatctttc
gcttgcacctgtttggcaagatctacgaccgcgcttccaagtacttcaa
gaccaagaagatgcgcatggcattcacgtttcagaccatgtatatgggc
atgtcgccctacgatgcgcctgctgtctacagcctgttgcagtacaccg
agttcgctgaaggcatctggtatccccgtggcggcttcaacatggtggt
tcagaagctagaggcgattgcaaagcaaaagtacgatgccgagtttatc
tacaatgcgcctgttgccaagattaacaccgatgatgccaccaaacaag
tgacaggtgtaaccttggaaaatggccacatcatcgatgccgatgcggt
tgtgtgtaacgcagatctggtctatgcttatcacaatctgttgcctccc
tgccgatggacgcaaaacacactggcttccaagaaattgacgtcttctt
ccatttccttctactggtccatgtccaccaaggtgcctcaattggacgt
gcacaacatctttttggccgaggcttatcaggagagctttgacgaaatc
ttcaaggactttggcctgccttctgaagcctccttctacgtcaatgtgc
cctctcgcatcgatccttctgctgctcccgacggcaaggactctgtcat
tgtcttggtgcctattggtcatatgaagagcaagacgggcgatgcttcc
accgagaactacccggccatggtggacaaggcacgcaagatggtgctgg
ctgtgattgagcgtcgtctgggcatgtcgaatttcgccgacttgattga
gcatgagcaagtcaatgatcccgctgtatggcagagcaagttcaatctg
tggagaggctcaattctgggtttgtctcatgatgtgcttcaggtgctgt
ggttccgtcccagcacaaaggattctaccggtcgttatgataacctatt
ctttgtgggtgcaagcacgcatcccggaactggtgttcccattgtcctt
gcaggaagcaagctcacctctgaccaagttgtcaagagctttggaaaga
cgcccaagccaagaaagatcgagatggagaacacgcaagcacctttgga
ggagcctgatgctgaatcgacattccctgtgtggttctggttgcgcgct
gccttttgggtcatgtttatgttcttttacttcttccctcaatccaatg
gccaaacgcccgcatcttttatcaataatttgttacctgaagtattccg
cgttcataactctaatgtcatttaa

SEQ ID NOs:5 and 6 are the 5′ and 3′ PCR primers for a 529 by fragment within SEQ ID NO:4.

SEQ ID NO:5: primer MO4641:

caatctgttgcctccctgc

SEQ ID NO:6: primer MO4642:

atcctttgtgctgggacgg

SEQ ID NO:7 is the DNA sequence of the heterologous crtZ-Dc open reading frame harbored by a Yarrowia lipolytica strain.

SEQ ID NO:7: crtZ-Dc gene

atgcttgcgttgtggaataccgggatcgtgctactgactatcatcatca
tggaaggggtggcaacgttcgcacacaagtacatcatgcacggctgggg
atggggctggcatcattcgcaccataccccgcgcaaaggggcgtttgag
cgtaacgatctctatgcggtggtgtttgcgctactggccattgcgctga
tttacgcgggcagcgaagggtactggccgcttcagtggattggcgcggg
aatgaccggctacggcgtgatctactttatcgttcacgatggtcttgtc
caccagcgctggccgttccgttacgtgccgcgccgcggctatctgcgcc
gcctctacatggcacaccggctgcatcacgcggtgcgggggcgcgaagg
gtgcgtctccttcgggtttatctacgccccaccggtggacaagctgcag
gcggtgctgcgcgaacgtaacggcagacccgccagcgcgggcgctgcca
gaggtgcggatcgcgcggcggccagctcgccttccgggaagccatcgcc
tgcttcgcgccggaaataa

SEQ ID NO:8 is the DNA sequence encoding PhoI nuclease from Pyrococcus horikoshii OT3, as optimized for expression in Yarrowia lipolytica, and includes restriction sites for cloning and a 5′ ribosomal binding site.

SEQ ID NO:8:

tgctagccacaaaaatggagatgtacaaggttggtcgatacctcgttga
ttccctccagatttacttccccgcttctcttgagatccaggaggagctt
attaacaacggcttctacgttccccgatctcctgatcgaaaggtttcta
tgcccattcccattgtttactccgattttggtggtcgagttatttctat
tgagcgactcattcctcccgagtggcttgagatttctcccgagcagctc
ggttgggaggagacttaccttgagaacaagcgaggtttcaagctcccta
aggaggaggtttacgttgatgtttctatttctaacgattccattatttt
cgagcttgacgttaagaactaccaccttgagcgaacctccattcgaggc
atcaaccctgagaagtggaagaactgggttatgttttacattgatctca
agtacgttgatgagttcattaacgctcttcgagagcacatccctgcttt
cgagaacaagacccgagttattcgagagaagcagcagggtggcaaggag
gttacttactacgctaaggttaacgtcaagaacttctctctttgtctcg
gttgtttcgatctcgctcagcgataccttcagatcaaggctaaggagca
ctgtaacatttaccctggttctcccacctgtaacgattctctctctaag
ctcaagctccgacttgagtacgatccctccattaccacctttgctaagg
ttggtattgccaagatttctggtaagcgaccccagatcatggttaagct
cacctctaccgagactaagaccattcgaggtattctcaagcctgagatt
aagggtaaggcccgaggtaagctcgtttactgtgatcaccgagagaagc
gacagtacattgctcttgacctcttcgatttttacaaggccctcgtttc
tactaagaagtacgagggtaagctccctaccgatgattaaacgcgt

SEQ ID NO:9 is the non-optimized DNA sequence encoding PhoI nuclease, from Pyrococcus horikoshii OT3.

SEQ ID NO:9:

atggaaatgtataaggttggaagataccttgttgattcccttcaaatata
ttttcccgcttcactagagatccaggaggagcttataaataatggcttct
acgtacccaggagcccagatagaaaagtaagcatgccaattccaatagta
tactccgattttggagggagggttatatctattgagaggttaatacctcc
ggagtggcttgaaatctcgccagagcaattaggttgggaagaaacttatt
tagagaacaaacgtggttttaaactacctaaagaagaggtctacgtggat
gttagtataagcaacgattccataatattcgagctagacgtgaaaaatta
tcaccttgaaaggacatccattagaggcatcaatcctgaaaaatggaaga
attgggtgatgttttatattgatttgaagtacgtagatgagttcattaac
gctttaagggaacatatcccagctttcgaaaataaaacaagggttattcg
tgaaaagcagcaagggggcaaggaagttacatattatgctaaagttaacg
tcaagaatttcagcttatgcttaggatgcttcgatctcgctcaaaggtat
cttcaaatcaaagctaaagagcactgtaacatatatccaggctcaccaac
atgtaacgattctctaagcaaattaaaattaaggcttgaatacgatccat
ccattactacctttgctaaagttggaatagccaagatctcaggcaagaga
ccccagatcatggttaaattaacctctacggaaactaaaactataagggg
aatacttaagccagaaataaaaggtaaggcgcgtggtaaacttgtatatt
gcgatcacagagaaaagagacagtatatagctttggacttgtttgatttt
tacaaggccttggtaagcactaagaagtatgaaggtaagctaccgactga
tgattaa

SEQ ID NO:10 is the amino acid sequence of PhoI nuclease, as deduced from SEQ ID NO:8.

SEQ ID NO:10:

MEMYKVGRYLVDSLQIYFPASLEIQEELINNGFYVPRSPDRKVSMPIPIV
YSDFGGRVISIERLIPPEWLEISPEQLGWEETYLENKRGFKLPKEEVYVD
VSISNDSIIFELDVKNYHLERTSIRGINPEKWKNWVMFYIDLKYVDEFIN
ALREHIPAFENKTRVIREKQQGGKEVTYYAKVNVKNFSLCLGCFDLAQRY
LQIKAKEHCNIYPGSPTCNDSLSKLKLRLEYDPSITTFAKVGIAKISGKR
PQIMVKLTSTETKTIRGILKPEIKGKARGKLVYCDHREKRQYIALDLFDF
YKALVSTKKYEGKLPTDD

SEQ ID NO:11 is the DNA sequence encoding TaqI nuclease from Thermus aquaticus, as optimized for expression in Escherichia coli, and includes restriction sites for cloning.

SEQ ID NO:11:

CCATGGCATCAACCCAAGCACAAAAAGCCCTGGAAACCTTCGAACGCTTC
CTGGCGAGCCTGGACCTGGAATCATACCAACAGAAATACCGTCCGATTAA
AACCGTGGAACAGGATCTGCCGCGTGAACTGAACCCGCTGCCGGACCTGT
ATGAACATTACTGGAAAGCACTGGAAGATAATCCGTCCTTTCTGGGCTTC
GAAGAATTTTTCGATCACTGGTGGGAAAAACGTCTGCGCCCGCTGGACGA
ATTTATTCGCAAATATTTCTGGGGCTGCAGCTACGCGTTTGTTCGTCTGG
GTCTGGAAGCGCGTCTGTATCGCACCGCCGTCAGCATCTGGACGCAATTT
CATTTCTGCTACCGCTGGAACGCCTCTTGTGAACTGCCGCTGGAAGCAGC
ACCGGAACTGGATGCACAGGGCATTGACGCTCTGATCCACACCAGTGGCA
GCTCTACGGGTATCCAAATCAAAAAAGAAACCTACCGTAGTGAAGCAAAA
TCCGAAAATCGTTTTCTGCGCAAACAGCGTGGTACGGCTCTGATTGAAAT
CCCGTATACCCTGCAAACGCCGGAAGAACTGGAAGAAAAAGCAAAACGTG
CTCGCGTCAACGGCGAAACCTACCGTCTGTGGGCGAAAGTGGCCCATCAC
CTGGATCGCCTGGAAAATGGTTTTGTGATTTTCCGTGAATCATACGTTAA
ATCGATCGAACTGTTCCTGCAGAAAAACGCCCCGACCCTGTCGGGCCTGA
TTCGTTGGGACCGTGTCGCACAAGAAGCACTGACCGCTCCGTGAGGTACC

SEQ ID NO:12 and 13 are the 5′ and 3′ PCR primers used to amplify taqI using pMB6736 as template for expression in Corynebacterium glutamicum. Primers include restriction sites for cloning.

SEQ ID NO:12: primer MO9315:

CACACACCATGGCTTCCACTCAGGCTCAG

SEQ ID NO:13: primer MO9316:

CACACAGGTACCTTAGGGGGCGGTGAGGGCCTCC

### BRIEF DESCRIPTION OF THE DRAWINGS

FIG. 1 shows the time course of DNA degradation activity in Y. lipolytica strains with or without the taqI nuclease gene.

FIG. 2 shows DNA degradation activity at different temperatures in Y. lipolytica strains with or without the taqI nuclease gene.

FIG. 3 shows the completeness of DNA degradation based on PCR result.

FIG. 4 shows the production of astaxanthin and its precursors in Y. lipolytica strains with and without the taqI nuclease gene.

FIG. 5 shows the production of zeaxanthin and its precursors in a Y. lipolytica strain which has the taqI nuclease gene.

FIG. 6 shows the time course for degradation of DNA in Y. lipolytica strains producing astaxathin and zeaxanthin in fermentors.

FIG. 7 shows the temperature range around the optimum temperature of the TaqI and PhoI nucleases, respectively, in Y. lipolytica.

FIG. 8 shows the nuclease activity of TaqI nuclease in E. coli expressing TaqI nuclease gene.

FIG. 9 shows the nuclease activity of TaqI nuclease in C. glutamicum expressing TaqI nuclease gene.

FIG. 10 shows the temperature range around the optimum temperature of the TaqI nuclease in C. glutamicum.

### DETAILED DESCRIPTION OF THE INVENTION

The present invention provides a method of degrading nucleic acids in vivo and/or in situ using a heterologous thermophilic nuclease. The method is intended to degrade the deoxyribonucleic acids of an organism, recombinant DNA in particular, and thus inactivating the biological activity of the deoxyribonucleic acids at any stage during the production of the end product(s). According to the invention, a heterologous thermophilic nuclease gene is introduced into the organism in which nucleic acids are intended to be degraded. The thermophilic nuclease is latent at the normal growth temperature of the organism, but can be activated by raising the temperature, preferably to a range around the optimum temperature of the thermophilic nuclease.

In the present invention, the thermophilic nuclease is used to inactivate the biological activity of the nucleic acids of the host organism. The term “inactivate the biological activity of nucleic acids” is considered to refer to the degradation of nucleic acids by the present invention. When nucleic acid is cleaved such that it can no longer accomplish the roles of replication, transcription or translation, it is considered to be biologically inactivated. Preferably, the nucleic acid is recombinant DNA. Preferably, the nucleic acid will be cleaved into units which are 500 base pairs or less.

In the present invention, the thermophilic nuclease is produced by the host cell rather than being added exogenously, and thus degrades the nucleic acids of the host cell in vivo and/or in situ. The term “in vivo” refers to the degradation of nucleic acids inside the host cell by the thermophilic nuclease produced by the cell, which may or may not be viable at the time of degradation. The term “in situ” refers to the degradation of nucleic acids by the thermophilic nuclease produced by the host cell after the cell wall of the host cell is broken (fully or partially). For example, an in situ degradation can be performed at the end of the fermentation process where cells are lysed to release their contents. Degradation can be performed immediately after the cells are lysed or after some additional processing of the lysed broth has already occurred.

In connection with the present invention, the term “nucleic acid” denotes single-stranded, double-stranded, or partially-double stranded deoxyribonucleic acid (DNA) or ribonucleic acid (RNA).

The term “gene”, as used herein, generally refers to a nucleic acid encoding a polypeptide, optionally including certain regulatory elements that may affect expression of one or more gene products (i.e., RNA or protein). For example, a thermophilic nuclease gene according to the invention refers to the open reading frame (ORF) encoding a polypeptide of the thermophilic nuclease, and also to nucleic acid sequences which encode the ORF of the thermophilic nuclease together with certain promoters which are placed upstream of the ORF to affect the expression of the nuclease gene, and/or a region for termination of transcription, and/or other regulatory elements.

The term “heterologous”, as used herein, refers to a gene or a polypeptide that does not naturally occur in the organism in which it is being expressed. It is understood that, where a heterologous polypeptide is to be expressed in a host cell, it will often be desirable to utilize nucleic acid sequences encoding the polypeptide that have been adjusted to accommodate codon preferences of the host cell and/or to link the encoding sequences with regulatory elements active in the host cell. For example, when the host cell is a Yarrowia strain (e.g., Yarrowia lipolytica), it will often be desirable to alter the gene sequence encoding a given polypeptide such that it conforms more closely with the codon preferences of such a Yarrowia strain. In certain embodiments, a gene sequence encoding a given polypeptide is altered to conform more closely with the codon preference of a species related to the host cell. Such embodiments are advantageous when the gene sequence encoding a given polypeptide is difficult to optimize to conform to the codon preference of the host cell due to experimental (e.g., cloning) and/or other reasons. In certain embodiments, the gene sequence encoding a given polypeptide is optimized even when such a gene sequence is derived from the host cell itself (and thus is not heterologous). For example, a gene sequence encoding a polypeptide of interest may be codon optimized for expression in a given host cell even though such a gene sequence is isolated from the host cell strain. In such embodiments, the gene sequence may be further optimized to account for codon preferences of the host cell. Alternately, the (non-heterologous) gene sequence might not be codon optimized but instead be linked to a regulatory element other than its own (whether that regulatory element comes from another gene in the host or from another species). Those of ordinary skill in the art will be aware of host cell codon preferences and will be able to employ the inventive methods and compositions disclosed herein to optimize expression of a given polypeptide in the host cell.

As used herein, a “host cell” means any cell type that is amenable to introduction of a nucleic acid construct or expression vector. The means of introduction of the nucleic acid construct or expression vector can be in the form of transformation, transfection, transduction, and the like. The term “host cell” encompasses any progeny of a parent cell that is not identical to the parent cell due to mutations that occur during replication. In preferred embodiments, the host cells produce one or more end products which are desired biological compounds or fine chemicals. It is preferred that the host cell is an industrial strain. The host cell can be a genetically modified cell or a natural cell which was initially not genetically modified. A genetically modified host cell refers to a host cell which has been modified, engineered, or manipulated, often to overexpress certain biological compounds or fine chemicals.

The host cell may be any cell useful in the recombinant production of biological compounds and/or fine chemicals in a prokaryote (bacteria or archaea) or a eukaryote.

The term “biological compounds” is known in the art and includes compounds which are found in nature in living matter. Examples of biological compounds include, but are not restricted to: proteins, polypeptides, amino acids, nucleic acids, nucleotides, carbohydrates, and lipids.

The term “fine chemical” is known in the art and includes compounds which are used in various branches of industry such as, for example but not restricted to, the pharmaceutical industry, the agriculture, cosmetics, food and feed industries. These compounds include organic acids such as, for example, tartaric acid, itaconic acid and diaminopimelic acid, lipids, saturated and unsaturated fatty acids (e.g., arachidonic acid), diols (e.g., propanediol and butanediol), aromatic compounds (e.g., aromatic amines, vanillin and indigo), carotenoids, vitamins and cofactors.

Higher animals have lost the ability to synthesize vitamins, carotenoids, cofactors and nutraceuticals and therefore need to take them in, although they are widely synthesized by other organisms such as bacteria. These molecules are either biologically active molecules per se or precursors of biologically active substances which serve as electron carriers or intermediates in a number of metabolic pathways. These compounds have, besides their nutritional value, also a significant industrial value as coloring agents, antioxidants and catalysts or other processing aids. The term “vitamin” is known in the art and includes nutrients which are required by an organism for normal functioning, but cannot be synthesized by this organism itself. The group of vitamins may include cofactors and nutraceutical compounds. The term “cofactor” includes non-protein compounds which are necessary for the occurrence of normal enzymatic activity. These compounds may be organic or inorganic; the cofactor molecules of the invention are preferably organic. The term “nutraceutical” includes food additives which promote health in organisms and animals, especially in humans. Examples of such molecules are vitamins, antioxidants and likewise certain lipids (e.g., polyunsaturated fatty acids).

Preferred fine chemicals or biosynthetic products which can be produced in organisms of the genus Yarrowia are: carotenoids such as, for example, phytoene, lycopene, beta-carotene, alpha-carotene, beta-cryptoxanthin, lutein, zeaxanthin, astaxanthin, canthaxanthin, echinenone, 3-hydroxyechinenone, 3′-hydroxyechinenone, adonirubin, violaxanthin, adonixanthin, and/or modified forms thereof (including, but not limited to isomerization, esterification, glycosylation, and breakdown products of these compounds); quinone derived compounds such as, for example, ubiquinone (including, but not limited to, coenzymes Q10 and Q5 to Q9 quinone compounds), vitamin K compound, and vitamin E compound; and retinolic compounds such as, for example, retinol, retinal, retinoic acid, and retinol palmitate.

The prokaryotic host cell may be any gram-positive or gram-negative bacterium. Gram-positive bacteria include, but are not limited to, Bacillus, Brevibacillus, Clostridium, Corynebacterium, Geobacillus, Lactobacillus, Lactococcus, Paenibacillus, and Streptomyces. Gram-negative bacteria include, but are not limited to Escherichia coli, Pseudomonas and Paracoccus.

The bacterial host cell may be any Bacillales cell including, but not limited to, Bacillus amyloliquefaciens, Brevibacillus brevis, Bacillus circulans, Bacillus clausii, Bacillus coagulans, Bacillus lentus, Bacillus licheniformis, Geobacillus stearothermophilus, Bacillus subtilis, and Bacillus thuringiensis cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The bacterial host cell may also be any Paracoccus cell including, but not limited to Paracoccus denitrificans, Paracoccus versutus, Paracoccus carotinifaciens, Paracoccus marcusii, and Paracoccus zeaxanthinifaciens cells.

The host cell may also be a eukaryote, such as a mammalian, insect, plant, algal, or fungal cell.

The algal host cell may be a Crypthecodinium, Schizochytrium, and Thraustochytrium cell such as a Crypthecodinium cohnii, Schizochytrium sp. or Thraustochytrium sp. cell.

The host cell may be a fungal cell. “Fungi” as used herein include the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

The fungal host cell may also be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeasts shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Schizosaccharomyces pombe or Yarrowia lipolytica cell.

Fungal cells may be transformed by a process involving protoplast formation, transformation of the protoplasts, and regeneration of the cell wall in a manner known to those of ordinary skill in the art. Suitable procedures for transformation of Aspergillus and Trichoderma host cells are described in EP 238023 and Yelton et al., 1984, Proc. Natl. Acad. Sci. USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163-168; and Hinnen et al., 1978, Proc. Natl. Acad. Sci. USA 75: 1929-1933.

For constructing a host cell strain with a heterologous thermophilic nuclease gene, the recombinant thermophilic nuclease gene construct is inserted into a host specific vector which allows optimal gene expression in the host. Vectors are well known in “Cloning Vectors” (Pouwels et al., Eds., Elsevier, Amsterdam-New York-Oxford, 1985). Vectors are to be understood as meaning not only plasmids, but all other vectors known to the skilled worker such as, for example, phages, viruses, such as SV40, CMV, baculovirus and adenovirus, transposons, IS elements, plasmids, cosmids, and linear or circular DNA. These vectors can be replicated autonomously in the host organism or chromosomally.

The vectors according to the invention allow the generation of recombinant organisms which are transformed, for example, with at least one vector according to the invention and which can be employed for producing the thermophilic nuclease. The above-described recombinant constructs according to the invention are advantageously introduced into the genome of the host organism and expressed. It is preferred to use usual cloning and transfection methods known to the skilled worker in order to bring about expression of the thermophilic nucleic acids in the expression system in question. Suitable systems are described, for example, in Current Protocols in Molecular Biology, F. Ausubel et al., Eds., Wiley Interscience, New York 1997.

In some embodiments, the modified host cell contains a plasmid carrying the heterologous thermophilic nuclease gene and the plasmid replicates autonomously in the host organism.

DNA is considered to be “recombinant” if it results from the application of Recombinant DNA Techniques. Examples of recombinant techniques include but are not limited to cloning, mutagenesis, transformation, etc. Recombinant DNA Techniques are disclosed, for example, in Sambrook, et al., Molecular Cloning: A Laboratory Manual, Cold Spring Harbor, N.Y. (1989) and in Ausubel F M, et al., Current Protocols in Molecular Biology, Wiley, New York (1998).

The term “nuclease” denotes an enzyme that effects hydrolytic cleavage of the ester bond between the 5′-phosphate group of a nucleotide and the 3′-hydroxy group of the adjacent nucleotide in a nucleic acid and therefore accomplishes the degradation of a DNA or RNA molecule. Nucleases are known from numerous organisms. Nucleases cleave either RNA or DNA to smaller units or even to their monomers.

In a preferred embodiment, the thermophilic nuclease according to the present invention is a DNA-degrading nuclease, and the nucleic acid which the thermophilic nuclease degrades is either double-stranded or single-stranded DNA. In a specific embodiment, the DNA being degraded is a recombinant DNA.

Although the preferred embodiment of the present invention relates to inactivation of DNA, inactivation of any nucleic acid molecule can be achieved. This includes inactivation of RNA, DNA-RNA hybrids, or nucleic acid-protein conjugates.

“DNA-degrading nuclease” means a nuclease which is able to cleave DNA molecules into smaller units or monomers. Such enzyme is also termed “DNase” in the state of art. “RNA-degrading nuclease” is a nuclease that is able to cleave RNA molecules into smaller units or monomers. Such enzymes are also termed “RNase” in the state of art.

The DNA or RNA nuclease can be an endonuclease or an exonuclease. An endonuclease is an enzyme that degrades a nucleic acid chain from a position inside the chain. Conversely, an exonuclease is an enzyme that degrades a nucleic acid chain from one or both ends of the chain. It is preferred for the nuclease to be an endonuclease.

“Thermophilic nuclease” means a nuclease which has its optimal activity at a temperature of 45° C. or higher. Said optimal activity is observed at a temperature that is optimal for the activity of the nuclease. The optimal temperature may be determined for the nuclease in an in vitro assay, or in an in vivo assay where the nuclease is expressed in the recombinant host according to the present invention, using a suitable substrate and under assay conditions (in terms of medium composition, assay time, etc.) that are relevant for the intended application, as determined by a person skilled in the art. As an example, the temperature optimum of a nuclease such as TaqI is typically determined in vitro using the purified TaqI nuclease. A fixed quantity of enzyme can be incubated with one microgram of lambda DNA in a fifty microliter reaction held at a range of temperatures (typically from 25 to 85° C.) for one hour. The optimum temperature is the one at which the most digestion is observed (for example, as assayed by gel electrophoresis of the resulting lambda DNA TaqI restriction fragments). Alternately, one unit of enzyme is defined as the amount of protein required to fully digest one microgram of lambda DNA in a fifty microliter reaction in one hour at the optimum temperature. Therefore, the optimum temperature is the temperature at which the quantity of protein required to achieve full digestion is the lowest. To define the optimum temperature within the cell, a defined ratio of enzyme to DNA substrate (as used in in vitro assays) cannot be guaranteed. Instead, a more pragmatic approach is taken. Given that the optimum temperature in vivo is likely to be similar to in vitro, the in vitro defined optimum can be used as a guide. Nuclease acid digestion by the nuclease is examined at and around the in vitro optimal temperature. The optimal temperature is the one at which the most digestion is observed. For some nucleases, the in vivo optimal temperature is a range of temperatures rather than a single point, as complete digestion of genomic DNA can be observed throughout the temperature range.

The thermophilic nucleases disclosed in the present application include both native thermophilic nucleases and any homologue or variant thereof which has thermophilic nuclease activity. Means to obtain thermophilic nucleases used in the present invention include but are not limited to: isolation from thermophiles, modification of a homologous nuclease to acquire thermophilic activity and/or increase thermotolerance by rational enzyme engineering and/or directed evolution. Unlike the majority of cells, thermophiles are heat-loving organisms with an optimum growth temperature of 50° C. or higher. In contrast, most cells grow at a lower temperature and cannot survive at the high temperature preferred by thermophiles. For example, common microorganisms grow at a temperature between 25 and 40° C., mammalian cell cultures grow at around 37° C., and plant and insect cultures grow at their respective low temperatures which are known in the art. Thermophilic nucleases lose activity outside their optimum temperature range and have lower activity at the temperature at which common microorganisms and cell cultures grow. The levels of nuclease activity of each thermophilic nuclease at a low temperature are different and thus have to be determined individually. In the present invention, a thermophilic nuclease is considered latent at a low temperature if the nuclease activity is reduced to less than 20% of the peak activity at the optimum reaction temperature of the nuclease.

The method of the invention is amenable to a wide range of thermophilic nucleases. Any nuclease which is latent at the normal growth temperature of the host microorganisms or eukaryotic cell cultures but is (highly) active at its own optimum reaction temperature is suitable for use in the present invention.

Some thermophilic nucleases are less suited for the present invention than others. For example, some thermophilic nucleases have low-level nuclease activities at low temperatures between 25 and 37° C. Such residual activities at the growth temperature of the host cell may constrain the growth of the host cells and thus hinder the production of their end product. Some thermophilic nucleases may not have strong nuclease activity at their optimum temperature and thus are inefficient in degradation of nucleic acids. Thus, a screening process may be performed to select those thermophilic nucleases which work best in the present invention.

The best thermophilic nuclease candidates are those whose nuclease activities are low enough at the normal growth temperature so as not to impose any significant stress on the host organism, and are high enough at the optimum reaction temperature to perform efficient degradation of the nucleic acids of the host.

Owing to the property of displaying nucleic acid degradation activity at a temperature that is outside the temperature range in which a normal host organism grows, the thermophilic nuclease according to the invention can be used advantageously in degrading nucleic acids of the host cell in a controlled manner. For example, in one embodiment, the present invention discloses that a heterologous thermophilic nuclease gene is introduced into a host cell and thus is used to degrade the nucleic acids of a host cell. According to the invention, the thermophilic nuclease gene is introduced into a host cell whose nucleic acids are to be degraded. The host cell is grown at its optimum growth temperature at which the heterologous thermophilic nuclease is latent. After the host cell is grown for a period of time to a certain stage, such as at the harvest time of the cell, the activity of the thermophilic nuclease in the host call can be activated by switching the temperature to one which is optimal for the nuclease activity. By activating the thermophilic nuclease, the DNA and/or RNA of the host cell are degraded by the nuclease, thus eliminating activities of the DNA and/or RNA of the host cell.

According to the present invention, any homologues or variants of a native thermophilic nuclease may be used in exchange for the native thermophilic nuclease as long as the homologue or variant has thermophilic nuclease activity.

In another embodiment, instead of using a heterologous thermophilic nuclease gene, the method according to the present invention could use a homologue or variant of a host cell nuclease which has been made thermophilic using methods such as rational enzyme engineering and/or directed evolution.

In one embodiment, one thermophilic nuclease is introduced into a host cell and thus is used to degrade the nucleic acids of the host cell. In another embodiment, two or more thermophilic nucleases are introduced into a host cell. The introduction of additional thermophilic nucleases may, for example, enhance the efficiency of the degradation of the nucleic acid.

In another embodiment, the sequence of the recombinant DNA to be degraded is optimized to include additional cleavage sites of the thermophilic nuclease. The addition of the cleavage sites helps to ensure the complete degradation of the recombinant DNA. The recombinant DNA to be degraded can include the nuclease gene itself. In a specific embodiment, the sequence of the recombinant DNA is engineered to include one cleavage site every 500 or fewer nucleotides. The cleavage site may be designed to be recognized by a single nuclease or multiple nucleases. In another specific embodiment, the sequence of the recombinant DNA is engineered to include cleavage sites in a manner in which the amino acid sequence of the resulting protein is not altered.

The above method of nucleic acid degradation can be carried out at any scale ranging from lab bench to commercial scale fermentation. As described in the background section of this application, DNA of the host cell, especially recombinant DNA, is preferred to be degraded prior to product recovery (for example, at the end of the fermentation process). The present invention provides a process for degrading the recombinant DNA in the biomass and thus inactivating any recombinant DNA. The biomass can be, for example, cells harvested from cultivation of microorganism or higher eukaryotic cell culture.

In a specific embodiment, the invention provides a fermentation process by which the recombinant DNA in the biomass is inactivated. The methodological steps for such process are exemplified as follows: First, the gene encoding the thermophilic nuclease according to the invention is introduced into the host cell. Integration of a nucleic acid construct containing the gene of a thermophilic nuclease into the host cell can take place intrachromosomally or extrachromosomally. Second, the host cell is grown in a fermentor at a temperature which is optimal for the growth of the host cell and/or the production process. Third, at the end of the fermentation process, the temperature of the fermentor is raised to the optimum temperature of the thermophilic nuclease and thus activates the nuclease. The higher temperature is maintained for a period of time to allow sufficient degradation of the DNA (recombinant or not) in the host cell. Subsequently, the biomass is collected and optionally is further processed to purify the end product (if not the biomass itself).

In another embodiment, instead of degrading DNA in the biomass, the method disclosed in the present invention can also be used for degrading DNA in partially purified products. For example, in the fermentation process described above, instead of activating the thermophilic nuclease before collection of biomass, the activation of the nuclease can be withheld until after the biomass is collected and further processed to obtain partially purified end products. The thermophilic nuclease is then activated to degrade the DNA contained in the partially purified end products, which can then be further purified, if desired. In the initial (partial) purification process, it is essential to maintain the activities of the nuclease. In fact, as long as the activity of the thermophilic nuclease is preserved during the post biomass collection process, the degradation of DNA of the host cell can be performed at any stage after the biomass is collected.

The process according to the invention can be used in many other technical fields in addition to fermentation. For example, the process according to the invention can be used for inactivating the DNA of the host cell during vaccine production.

Cell culture-based methods of producing vaccine containing virus or viral antigens are increasingly used in commercial production of viral vaccines. However, it is known in the art that one of the risks associated with using cell culture for vaccine production is the exposure of vaccine recipients to contaminating residual cellular DNA of the host cell. There is a concern that any host cell DNA be removed from the final formulation of a vaccine manufactured in the host cell system, thus eliminating any potential oncogene from the final product. The process according to the invention can be carried out to degrade the host cell nucleic acid associated with a virus or a viral antigen thereof produced by cell culture. The process comprises the steps of: (a) introducing the gene of a thermophilic DNA nuclease into a host cell culture and thus creating a new host strain; (b) inoculating the population of the new host strain with a virus; (c) culturing the population of host cells so as to allow the virus to replicate, wherein the culturing is conducted at a temperature at which the thermophilic nuclease is latent; (d) degrading the DNA of the host cells by changing the temperature in step (c) to a temperature at which the thermophilic DNA nuclease is active; (e) collecting the produced virus thereby providing a viral harvest, and (f) isolating the virus. The order of steps (d) through (f) can be exchangeable. The virus can be, for example, influenza virus. However, any virus or viral antigen can be produced according to the invention.

The process according to the invention can also be used for inactivating the nucleic acid of a genetically modified crop. For example, a thermophilic nuclease according to the invention can be introduced into a genetically modified crop. The crop is grown in the field at temperatures which normally do not exceed 50° C. After the crop is harvested, it can be, for example, processed at the optimal temperature of the above thermophilic nuclease in order to degrade the crop nucleic acid.

The process according to the invention can also be used for reducing viscosity in a cell lysate. It is known in the art that when cells are lysed, DNA is released from the cell and thus causes the cell lysate to become viscous. This hinders the further processing of cell lysate. The thermophilic DNA degrading nuclease works by cleaving DNA into small oligonucleotide fragments and thus removing the viscosity caused by the DNA content in a controlled manner.

The thermophilic nuclease that is used according to the invention can be, for example, TaqI endonuclease from Thermus aquaticus. TaqI has an optimum temperature of 65° C. Other thermophilic nucleases can be tested for their suitability of being used in the present invention. Examples of additional thermophilic nucleases include: Tsp509I which has an optimum temperature of 65° C., MwoI which has an optimum temperature of 60° C., PhoI which has an optimum temperature of 75° C., BsaJI which has an optimum temperature of 60° C., and BspQI which has an optimum temperature of 50° C.

Basically, any existing thermophilic nuclease known in the state of art or any thermophilic nuclease yet to be identified can be used according to the invention, as long as such thermophilic nuclease has high nucleic acid cleavage activity at its optimum reaction temperature but is latent at the lower temperature at which the host microorganisms or cell cultures normally grow.

Within the scope of the present invention it is preferred that the temperature for degrading nucleic acid is the temperature known to have the optimal activity of the specific thermophilic nuclease. For example, one preferred temperature for degrading nucleic acids of the host cell using thermophilic nuclease TaqI is 65° C. because TaqI is known to be most active at 65° C. However, the optimum temperature according to the present invention is not limited to a single temperature point. According to the present invention, it is also preferred that the temperature for degrading nucleic acids is any temperature which is within a range of ±5° C. of the known optimum temperature of the thermophilic nuclease. In the case of TaqI, for example, a preferred temperature is any temperature which falls between 60° C. and 70° C. In another example, PhoI is known to be most active at 75° C. A preferred temperature for PhoI is any temperature which falls between 70° C. and 80° C. It is particularly preferred that a temperature which falls within ±1° C., ±2° C., ±3° C., or ±4° C. of the known optimum temperature of the thermophilic nuclease is the temperature for activating the thermophilic nuclease. Thus, in one embodiment, the temperature used for activating the thermophilic nuclease activity is within ±5° C. of the known optimum temperature of the thermophilic nuclease.

It is clear to one skilled in the art that the temperature used for activating the thermophilic nuclease activity is not only limited to a temperature range that is within ±5° C. of the known optimum temperature of a selected thermophilic nuclease. As an alternative, the temperature used for activating the thermophilic nuclease activity can be any temperature at which the host cell does not actively grow but the thermophilic nuclease has active enzyme activity. For example, the temperature used for activating TaqI can be at 75° C. where the host cell does not have active growth but the thermophilic nuclease still has enzyme activity. In one embodiment, the temperature used for activating the thermophilic nuclease activity is a temperature or temperature range at which the thermophilic nuclease has 20% or more of its optimal enzyme activity. In another embodiment, the temperature used for activating the thermophilic nuclease activity is a temperature or temperature range at which the host cell grows at 20% or less of its optimal growth rate or has no growth and the thermophilic nuclease has 20% or more of its optimal enzyme activity. In another embodiment, the temperature used for activating the thermophilic nuclease activity is a temperature between 40° C. and 100° C.

To the extent where the degradation of nucleic acids can be performed in a range of temperatures without jeopardizing its efficiency, a preferred temperature is any temperature within the temperature range that strikes the optimum balance with other considerations, including but not limited to end-product titer, end-product stability, end-product purity, end-product yield, ease of handling of the material for subsequent processing steps, etc.

Thus, it is within the scope of the present invention that any temperature which permits efficient degradation of the nucleic acids may be chosen to activate the thermophilic nuclease activity. Sometimes such temperature may not provide the best thermophilic nuclease activity, but in combination with some additional factors, it may provide best overall efficiency. For example, heat pasteurization may be used in place of the heat activation step of thermophilic nuclease. By heating up the biomass gradually during pasteurization, both pasteurization of the host cell and activation of thermophilic nuclease activity in the cell can be performed in one step, and thus result in cost savings. In such case, the optimum temperature or temperature range is crossed on the trajectory to the pasteurization temperature.

The nucleic acid degradation process according to the present invention can be carried out relatively quickly. According to an embodiment of the invention, the process of nucleic acid degradation is carried out at the optimum temperature of the thermophilic nuclease for the duration of less than 48 hours, less than 24 hours, less than 1 hour, less than 40 minutes, less than 30 minutes, or less than 20 minutes. The optimal time length can be determined experimentally for each thermophilic nuclease and under specific experimental conditions.

The present invention can be used in any host cell. Examples of host cells include microorganisms and higher eukaryotic cells such as agricultural crops or animal cells. According to the invention, the microorganisms can be for example prokaryotic cells, such as bacteria or archaebacteria, or eukaryotic cells, such as yeasts, lower or higher fungi, algae, or protozoa. According to a preferred embodiment, microorganisms are bacterial cells, fungal cells or yeast, or microalgal cells.

In certain embodiments of the invention, the organism whose nucleic acids can be degraded according to the method of the present invention is a microorganism from a genus such as, but not limited to, Yarrowia, Bacillus, Corynebacterium, Escherichia, Pseudomonas, Paracoccus, Candida, Hansenula, Saccharomyces, Mortierella, Schizosaccharomyces, Aspergillus, Fusarium, Trichoderma, Crypthecodinium, Schizochytrium, and Thraustochytrium.

In one embodiment of the invention, the host cell in accordance with the present invention is Yarrowia lipolytica. Advantages of Y. lipolytica include, for example, tractable genetics and molecular biology, availability of genomic sequence, suitability to various cost-effective growth conditions, ability to grow to high cell density, and history of safe use. There is already extensive commercial experience with Y. lipolytica.

Escherichia coli is useful because it is the most commonly used bacterial strain. It has been regularly used as a carrier strain for recombinant DNA.

Saccharomyces cerevisiae is also a useful host cell in accordance with the present invention, particularly due to its experimental tractability and the extensive experience that researchers have accumulated with this organism.

The host cell in accordance with the present invention also includes higher eukaryotic cells. The term “higher eukaryotic cell” means a eukaryotic cell of a high state of development, such as those which occur for example in animal or plant organisms. Higher eukaryotic cells do not perform all vital biochemical and metabolic functions independently and thus may be grown as cell culture. The higher eukaryotic cell in accordance with the present invention comprises, inter alia, all cells from a mammal, a plant, an insect, or an avian. According to one embodiment of the invention, the higher eukaryotic cell is from a mammal. According to another embodiment of the invention, the higher eukaryotic cell is from a plant. According to another embodiment of the invention, the higher eukaryotic cell is from an insect.

According to one embodiment of the invention, the degradation of the nucleic acids of the host cell is carried out with no or minimal impact to the other cellular components of the host cell. In a preferred embodiment, the degradation of the nucleic acids of the host cell is carried out with no or minimal impact to the production of the biological compounds and/or fine chemicals which the host cell is engineered to produce. In one embodiment, the host cell is a Yarrowia strain and the biological compounds which are genetically engineered to produce include: phytoene, lycopene, beta-carotene, alpha-carotene, beta-cryptoxanthin, lutein, zeaxanthin, astaxanthin, canthaxanthin, echinenone, 3-hydroxyechinenone, 3′-hydroxyechinenone, adonirubin, violaxanthin, adonixanthin, ubiquinone, vitamin K, vitamin E, retinol, retinal, retinoic acid, retinol palmitate, and/or modified forms thereof (including, but not limited to isomerization, esterification, glycosylation, and breakdown products of these compounds). In one embodiment, the Yarrowia strain is a genetically modified Yarrowia strain. In a specific embodiment, the genetically modified Yarrowia strain cell introduced with a thermophilic nuclease gene is Yarrowia lipolytica strain ML12924 and the biological compound produced is astaxanthin. In another embodiment, the genetically modified Yarrowia strain cell introduced with a thermophilic nuclease gene is Yarrowia lipolytica strain ML12921 and the biological compound produced is zeaxanthin. In another embodiment, the genetically modified Yarrowia strain cell introduced with a thermophilic nuclease gene is Yarrowia lipolytica strain ML12805 and the biological compound produced is lycopene.

In one embodiment of the invention, codon optimization is performed in order to enhance the expression of the heterologous nuclease gene in the host cell. It is known in the art that the expression of non-native genes is hampered by the existence of variation in their respective codon usage pattern compared to the host organism. To overcome these problems, codon optimization according to the present invention is performed on the thermophilic nuclease gene in order to match the host codon usage before the gene is introduced into the host cell. This process encompasses the replacement of rare codons within the DNA sequence of the nuclease gene in order to closely match the host codon usage bias while retaining 100% identity or near 100% identity to the original amino acid sequence. This process of codon optimization also allows for the simultaneous modification of predicted mRNA secondary structures that could result from changes in the GC content.

In the thermophilic nuclease of the present invention, the codon usage pattern is altered from that typical of the thermophilic nuclease gene to modify the codons without altering the encoded amino acid sequence. The nucleic acid sequences for many thermophilic nuclease genes are known. In cases where the sequence is not initially known, the information can be derived by methods known to those of ordinary skill in the art (including, but not limited to DNA and RNA sequencing). In accordance with this invention, thermophilic nuclease gene segments were converted to sequences having identical translated sequences but with alternative codon usage.

In one embodiment of the invention, the codon usage pattern of the native thermophilic nuclease TaqI (SEQ ID NO:2) is optimized to the codon bias of Y. lipolytica. The resulting codon modified TaqI gene is specified in SEQ ID NO:1. In another embodiment, the codon usage pattern of the native thermophilic nuclease TaqI (SEQ ID NO:2) is optimized to the codon bias of E. coli. The resulting codon modified TaqI gene is specified in SEQ ID NO:11. The amino acid sequence encoded by SEQ ID NO:1, SEQ ID NO:2, and SEQ ID NO:11 is specified in SEQ ID NO:3.

In one embodiment of the invention, the codon usage pattern of the native thermophilic nuclease PhoI (SEQ ID NO:9) is optimized to the codon bias of Y. lipolytica. The resulting codon modified phoI gene is specified in SEQ ID NO:8. The amino acid sequence encoded by SEQ ID NO:8 and SEQ ID NO:9 is specified in SEQ ID NO:10.

The codon modified taqI and phoI genes illustrated in SEQ ID NO:1 and SEQ ID NO:8 serve only as examples of many possible codon modified taqI and phoI genes, or other thermophilic nuclease genes. It is clear to a skilled person that any suitable codon modification method could be used to modify the thermophilic nuclease gene according to the present invention, and the resulting sequence from using different codon modification methods may vary. Having now generally described this invention, it will become more readily understood by referencing the following specific examples which are included herein for purposes of illustration only and are not intended to be limiting unless otherwise specified.

The following examples illustrate the invention.

### EXAMPLES

Table 1 below describes certain Yarrowia lipolytica strains used in the following exemplification:

TABLE 1
Yarrowia lipolytica strains.

| Strain | | |
|---|---|---|
| Number | Genotype | How Constructed |
| ML8195 | MATA erg9-4789::ura3 | Classical and standard |
| | HMG-tr GGS carB | molecular genetic techniques |
| | carRP(E78G) ura3 ade1 | |
| ML12805 | ML8195 [pMB6722] | Untargeted transformation |
| ML11956 | MATB erg9- | Classical and standard |
| | 4789::URA3HMG-tr GGS | molecular genetic techniques |
| | carB carRP crtW crtZ | |
| | (prototrophic) | |
| ML12924 | ML11956 [pMB6736] | Untargeted transformation |
| ML11218 | MATB erg9-4789::URA3 | Classical and standard |
| | HMG-tr GGS carB carRP | molecular genetic techniques |
| | crtW crtZ-Xa crtZ-Dc | |
| | crtW-Δ6180 | |
| ML12921 | ML11218 [pMB6736] | Untargeted transformation |
| ML13270 | ML11218 [pMB6868] | Untargeted transformation |

Yarrowia strains ML8195, ML12805, ML11956, ML12924, ML11218, ML12921 and ML13270 were constructed by the introduction of heterologous genes under the control of constitutive promoters (for example, TEFL), coupled with several generations of crossbreeding, starting with ML350 and ATCC201249 as described in U.S. Pat. No. 7,851,199 B2. The GGS gene and the truncated HMG gene (“HMG-tr”) were derived from Yarrowia lipolytica sequences corresponding to native geranylgeranyl pyrophosphate synthase and hydroxymethylglutaryl-CoA reductase genes, respectively. The carRP and carB genes were derived from Mucor circinelloides, and they encode a bifunctional phytoene synthase/lycopene cyclase and a phytoene dehydrogenase, respectively. The crtW gene was synthesized to encode the carotene ketolase of Parvularcula bermudensis. The crtZ gene was amplified from Xanthobacter autotrophicus (Xa), or synthesized to encode the carotene hydroxylase from Enterobacteriaceae bacterium DC404 (Dc) (SEQ ID NO:7). These genes are sometimes but not always associated with auxotrophic markers (URA3, LEU2, URA2, LYS1, ADE1) or a loxP site, remnant of a Hyg^{R} (hygromycin resistance) marker.

### Example 1

### Production of Plasmids for Yarrowia Strain Construction

Plasmids were generated for expression of TaqI thermophilic nucleases in Yarrowia as described in Table 2. A codon optimized taqI nuclease ORF sequence was synthesized de novo based on the sequence of the taqI nuclease gene of Thermus aquaticus (SEQ ID NO:2), using the Y. lipolytica codon bias as specified in SEQ ID NO:1. This codon-optimized taqI nuclease ORF was cleaved using NheI and MluI and ligated to pMB5082 cut with NheI and MluI to produce pMB6722. The resulting encoded TaqI protein of pMB6722 is specified in SEQ ID NO:3.

In a second plasmid, the same taqI nuclease gene sequence was excised from plasmid pMB6722 and cloned into a different plasmid backbone, pMB6157, to produce a new plasmid, pMB6736.

A plasmid was generated for expression of PhoI thermophilic nuclease as described in Table 2. A codon optimized phoI nuclease ORF sequence was synthesized de novo based on the sequence of the phoI nuclease gene of Pyrococcus horikoshii OT3 (SEQ ID NO:9), using the Y. lipolytica codon bias as specified in SEQ ID NO:8. This codon-optimized phoI nuclease ORF was cleaved using NheI and MluI and ligated to pMB6771 cut with NheI and MluI to produce pMB6868. The resulting encoded PhoI protein of pMB6868 is specified in SEQ ID NO:10.

Plasmid constructions were performed based on basic molecular biology and DNA manipulation procedures. All basic molecular biology and DNA manipulation procedures described in this and other examples are generally performed according to Sambrook et al. or Ausubel et al. (Sambrook J, Fritsch E F, Maniatis T (eds). 1989. Molecular Cloning: A Laboratory Manual. Cold Spring Harbor Laboratory Press: New York; Ausubel F M, Brent R, Kingston R E, Moore D D, Seidman J G, Smith J A, Struhl K (eds). 1998. Current Protocols in Molecular Biology. Wiley: New York).

TABLE 2
Plasmids

| Plasmid | Backbone | Insert | Oligos or source |
|---|---|---|---|
| pMB6722 | pMB5082 (URA3) | taqI | Synthesized |
| | | | NheI-MluI |
| | | | fragment |
| pMB6736 | pMB6157 (HygR) | taqI | NheI-MluI |
| | | | fragment from |
| | | | pMB6722 |
| pMB6868 | pMB6771 (HygR) | phoI | Synthesized |
| | | | NheI-MluI |
| | | | fragment |

### Example 2

### Demonstration of Nuclease Activity of Y. lipolytica Expressing TaqI Nuclease

In this example, the gene encoding TaqI nuclease was introduced into a host strain of Y. lipolytica. The TaqI nuclease activity in the host strain was tested.

Plasmid pMB6722 was cleaved with XbaI and transformed into host strain ML8195 (Table 1). Yarrowia strain ML8195 is a strain which was previously genetically modified to produce lycopene. Transformants introduced with the heterologous taqI nuclease gene were selected on YNB glutamate plates supplemented with 0.12 mM adenine; one such colony was selected and designated as Yarrowia strain ML12805. ML12805 was incubated for 3 days at 30° C. at 250 rpm in 20 mL YPD medium in a 125 mL flask. The cultures were split and 1.5 mL aliquots incubated at 65° C. or 25° C. for 5 min to 24 hr.

DNA from strains ML8195 and ML12805 was extracted and analyzed after incubation at 65° C. or 25° C. DNA was extracted using a “Smash and Grab” yeast genomic DNA extraction protocol. Essentially, 1.5 mL of a 3-day culture was pelleted, supernatant decanted and the cells resuspended in 0.5 mL water. Cells were pelleted for 10 seconds and supernatant decanted. The pellet was resuspended in 0.2 mL Smash and Grab Solution (1% SDS, 2% Triton-X100, 100 mM NaCl, 10 mM Tris pH 8.0, 1 mM EDTA) and 0.2 mL phenol-chloroform-isoamyl alcohol, and 0.3 g glass beads were added. The tubes were vortexed for 5 min using a multi-tube holder. To the mixtures, 0.2 ml, TE pH 8.0 (10 mM Tris, 1 mM EDTA) was added and centrifuged for 5 min. The aqueous phase was transferred to a new tube and 1.0 mL cold ethanol was added and mixed to precipitate the DNA. After centrifugation for 5 min, the supernatant was removed. The pellet was resuspended in 0.4 mL TE with 75 μg/mL RNaseA and incubated at room temperature for 5 min. 10 μL 4 M ammonium acetate and 1.0 mL ethanol was added. After incubation on ice for 5 minutes and centrifugation for 10 minutes, the supernatant was discarded. The pellet was washed with 0.5 mL 70% ethanol and centrifuged for 10 min. The supernatant was discarded and the pellet air dried, then resuspended in 100 μL TE. The resulting DNA from the above extraction process was analyzed by electrophoresis on 0.8% agarose gel in 1× Tris-acetate-EDTA (TAE) running buffer at 100 V for 1.5 h.

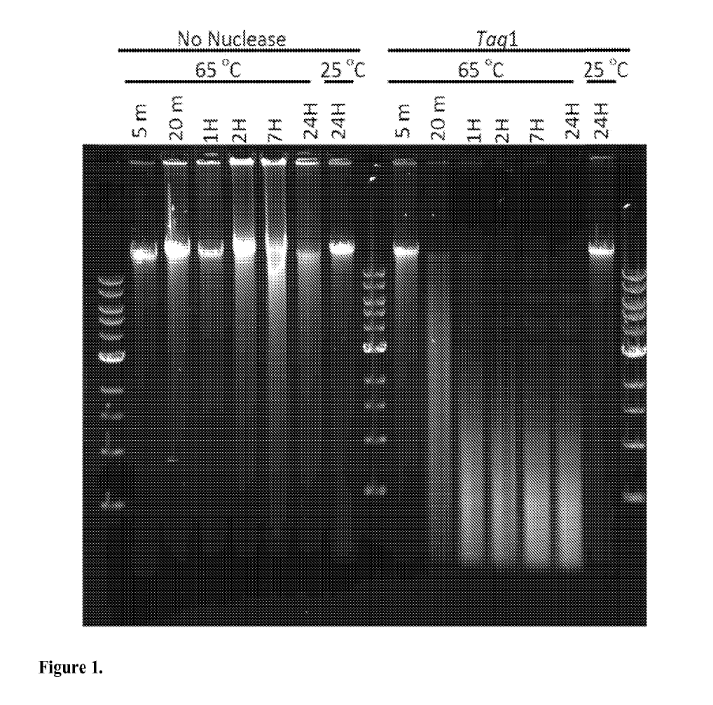

As shown in FIG. 1, the host strain of Y. lipolytica with no taqI nuclease gene (ML8195) did not display DNA degradation activity at either 65° C. or 25° C. The host strain of Y. lipolytica that harbors the taqI nuclease gene (ML12805) displayed DNA degradation activity at 65° C. but not at 25° C. The degradation activity was observed as early as 20 minutes after the cell culture was incubated at 65° C. The ML12805 strain did not display any DNA degradation activity at 25° C. even after being incubated for 24 hours, showing that TaqI nuclease in the Y. lipolytica strain is latent at 25° C.

### Example 3

### Temperature Activity Profile of TaqI

In this example, the activity of TaqI nuclease was examined at different temperatures.

Y. lipolytica strains ML12805 and ML8195 were grown for 3 days as described in Example 2. 1.5 mL aliquots of ML12805 and ML8195 cells were incubated at temperatures of 25° C., 30° C., 40° C., 50° C. and 65° C. for 1.5 h and DNA was extracted and analyzed as described in Example 2.

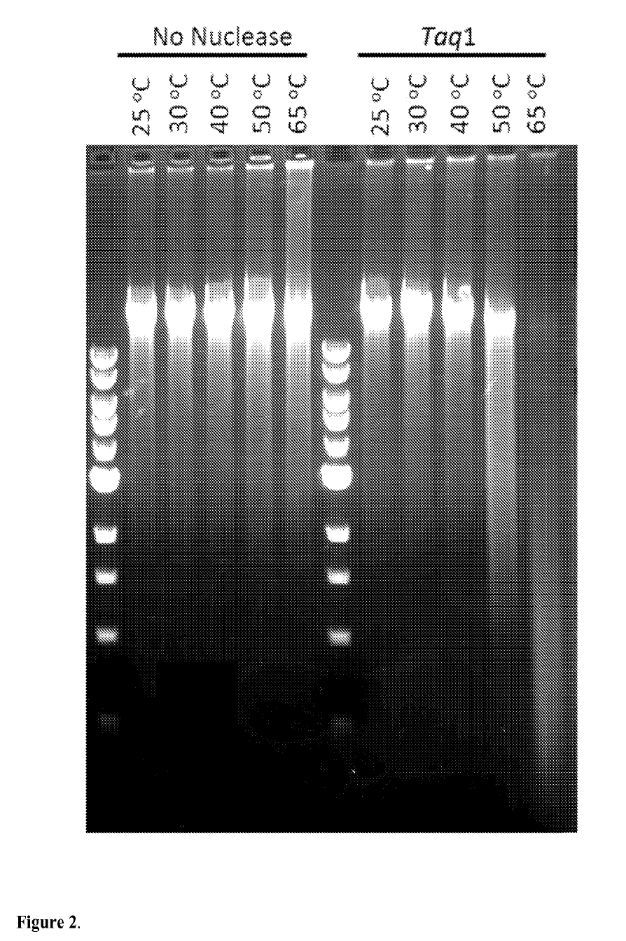

As shown in FIG. 2, no DNA degradation was observed at temperatures of 25° C., 30° C., 40° C., and 50° C., in both strains ML12805 and ML8195. DNA degradation was observed at 65° C., only in strain ML12805, as was shown in Example 2. The results show that TaqI nuclease is latent at temperatures equal to or below 50° C.

### Example 4

### PCR Analysis of In Vivo TaqI-Digested gDNA

In this example, the degradation of the heterologous carB gene in a recombinant Y. lipolytica strain was examined.

The carB gene is 1740 nucleotides in length and encodes phytoene dehydrogenase, an enzyme in the carotenoid biosynthetic pathway. The carB gene has been introduced into Y. lipolytica to enable production of carotenoids. As described in Example 2, ML8195 is a lycopene-producing strain lacking any thermophilic nuclease, and ML12805 is a derivative of ML8195 containing the taqI nuclease gene; both strains therefore contain the carB gene.

The presence of the carB gene was examined in strains ML8195 and ML12805 at both 25° C. and 65° C. Serial dilutions of genomic DNA from ML12805 incubated at 25° C. or 65° C. for 24 hours were prepared. PCR was performed on the above samples using primers designed for amplifying a 529 by fragment of the carB gene. The sequence of primer MO4641 is specified in SEQ ID NO:5. The sequence of primer MO4642 is specified in SEQ ID NO:6. The DNA fragment of the carB gene between oligos MO4641 and MO4642 contains two TaqI sites.

PCR was performed under the conditions described below. In each 25 μL reaction, 1 μL of diluted gDNA was combined with 0.5 μL of each primer (10 μM stock solution), 0.5 μL water and 22.5 μL of Platinum Taq Super Mix. The reaction parameters were 1 cycle at 94° C. for 2 min followed by 45 cycles at 94° C. for 30 s, 58° C. for 30 s and 72° C. for 60 s. A final single cycle of 72° C. for 5 min finished the PCR in a MJ Research PTC-225 Peltier Thermal Cycler. The entire reaction plus 1× loading dye was loaded onto a 2% agarose in 1×TAE gel with 0.5 μg/ml ethidium bromide and run at 100 V for 1.5 h.

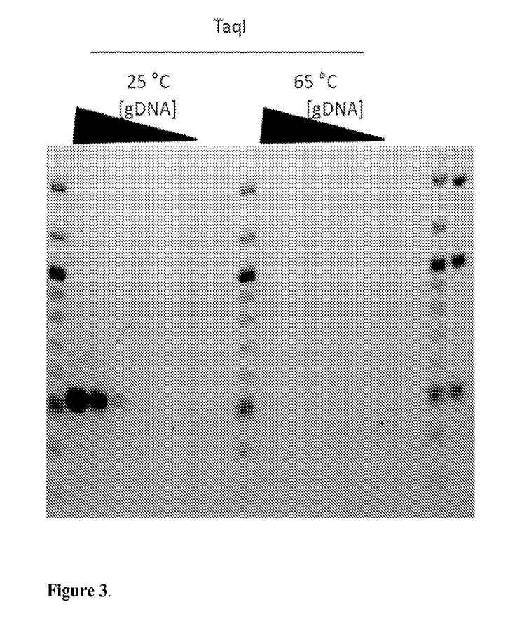

As shown in FIG. 3, for the Y. lipolytica strain in which the taqI nuclease gene was introduced (ML12805), a 529 by PCR product of carB was observed in the sample which was incubated at 25° C. but not in the sample which was incubated at 65° C. (serial dilutions of the PCR template were performed to demonstrate the sensitivity of the assay, as is known to those of ordinary skill in the art). These results show that the TaqI nuclease degraded the recombinant DNA (carB gene) in strain ML12805 when incubated at 65° C. FIG. 3 also illustrates that foreign DNA in strain ML12805 can be targeted for degradation, and that the cleavage is complete, resulting in no detectable fragment of approximately 500 nucleotides.

### Example 5a

### Astaxanthin Production in a Y. lipolytica Strain Harboring the taqI Thermophilic Nuclease Gene

In this example, the production of astaxanthin in a strain of Y. lipolytica was examined, both before and after the taqI nuclease gene was introduced.

In this example, a strain of Y. lipolytica which produces astaxanthin (ML11956) was used. A second strain (ML12924) was constructed by introducing the taqI nuclease gene (MB6736) into strain ML11956. The amount of astaxanthin and its precursors in both strains was measured. Measurements were made at different time points during the cell growth period and after the host strains were incubated at 65° C.

Strains ML11956 and ML12924 were grown in a fermentor using a fed-batch process. Samples were taken at different time points during the fermentation process and carotenoid analysis was performed according to the methods described previously in U.S. Pat. No. 7,851,199 B2.

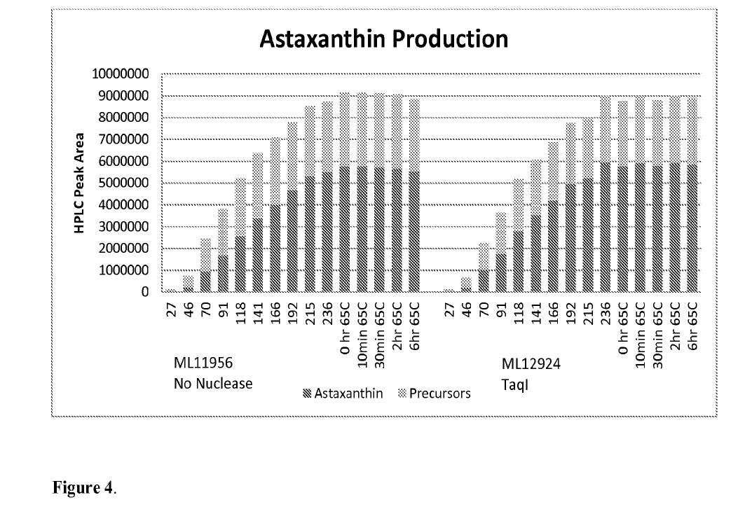

FIG. 4 shows that the amount of astaxanthin increased as strain ML11956 grew and the production eventually reached a plateau. The amount of astaxanthin did not change after ML11956 was incubated at 65° C. Strain ML12924 behaved in the same manner as strain ML11956. The amount of astaxanthin produced by strain ML12924 was the same as in strain ML11956 throughout the fermentation period and after incubation at 65° C. The amount of astaxanthin precursors produced also remained unchanged in strains ML11956 and ML12924 before and after incubation at 65° C. This result shows that neither the introduction of the taqI nuclease gene nor activation of its activity affected the amount or purity of astaxanthin produced by Y. lipolytica.

### Example 5b

### Zeaxanthin Production in a Y. lipolytica Strain Harboring the taqI Thermophilic Nuclease Gene

In this example, the production of zeaxanthin in a host strain of Y. lipolytica with the taqI nuclease gene was examined.

In this example, a strain of Y. lipolytica which produces zeaxanthin (ML11218) was transformed with a taqI nuclease gene (MB6736). In the resulting Y. lipolytica strain (ML12921), the amount of zeaxanthin and its precursors produced were measured. Measurements were made at different time points during the cell growth period and were also made after the host strain was incubated at 65° C.

Strain ML12921 was grown in a fermentor using a fed-batch process. Samples were taken at different time points during the fermentation process and carotenoid analysis was performed according to the methods described previously in U.S. Pat. No. 7,851,199 B2.

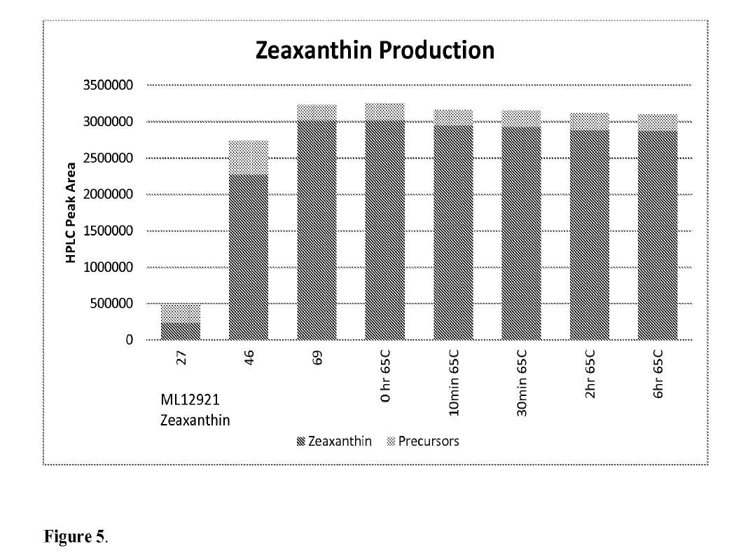

FIG. 5 shows that the amount of zeaxanthin increased as strain ML12921 grew and the production eventually reached a plateau. The amount of zeaxanthin produced did not change after the host cells were incubated at 65° C. The amount of zeaxanthin precursors also remained the same after the host cells were incubated at 65° C. In the control strain ML11218 (not shown), zeaxanthin production was similar to ML12921 in all the conditions tested. This result shows that neither the presence of the TaqI nuclease nor its activation affect the production or purity of zeaxanthin in Y. lipolytica.

### Example 5c

### Demonstration of TaqI Nuclease Activity in Strains Producing Astaxanthin or Zeaxanthin

In this example, the TaqI nuclease activities of Y. lipolytica strains ML11956, ML12924, and ML12921 described in Examples 5a and 5b were examined.

Strains ML11956, ML12924, and ML12921 were grown in fermentors. For strains ML11956 and ML12924 (producing astaxanthin in a longer fermentation), samples were taken at 46, 121 and 236 h post inoculation. For strain ML12921 (producing zeaxanthin in a shorter fermentation), samples were taken at 46 and 69 h post inoculation. At the end of the fermentation process (236 h for astaxanthin strains and 69 h for zeaxanthin strains) the temperature was raised to 65° C. and strains were incubated at this temperature for 6 hours. Samples were taken at 0 min, 10 min, 30 min, 2 h and 6 h after the shift to the higher temperature. DNA was extracted and analyzed as described in Example 2.

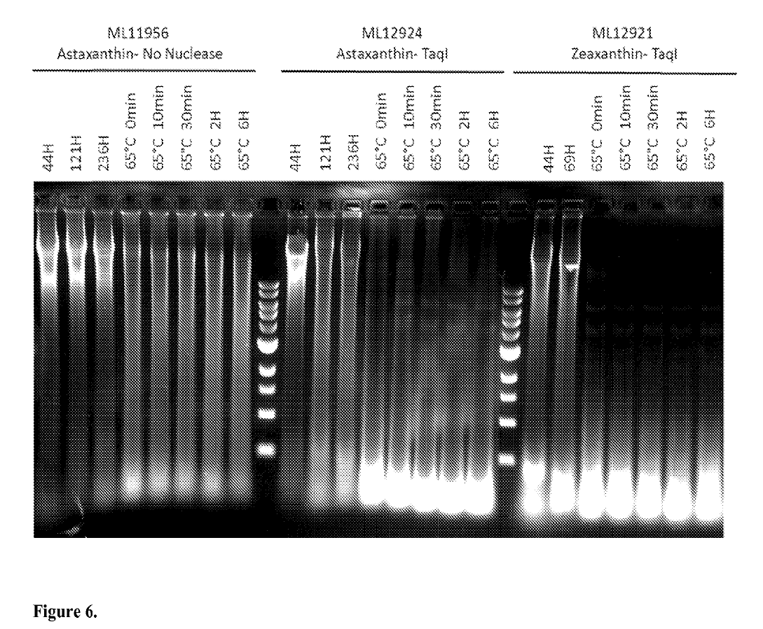

As shown in FIG. 6, no DNA degradation was observed in strain ML11956 lacking the nuclease. In both strains ML12924 and ML12921, DNA degradation was observed after the cells were incubated at 65° C. The above result shows that the TaqI nuclease was latent at the fermentation temperature but was active at 65° C. in Y. lipolytica strains, allowing for DNA degradation without affecting the amount or purity of the desired end product (astaxanthin or zeaxanthin).

### Example 6

### Temperature Range for TaqI and PhoI Centered Around their Respective Optima

In this example, the activities of TaqI and PhoI nucleases were examined at and around their optimum temperatures.

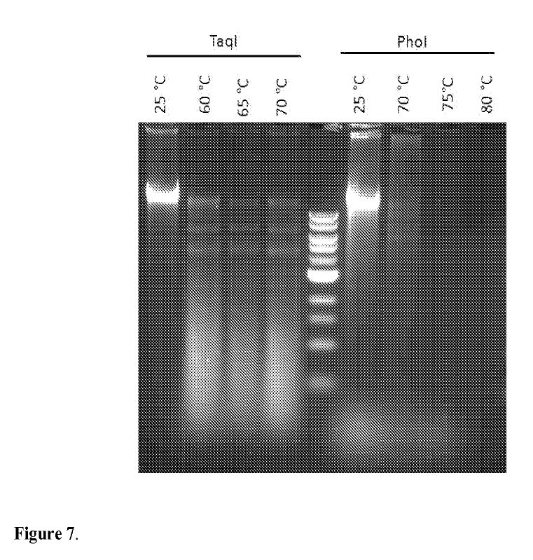

As shown in FIG. 7, the strain of Y. lipolytica harboring the taqI nuclease gene (ML12921) displayed DNA degradation activity at 65° C. but not at 25° C. ML12921 further displayed DNA degradation activity at 60° C. and 70° C., i.e., ±5° C. of 65° C., the optimal temperature for TaqI nuclease activity. Similarly, the host strain of Y. lipolytica with the PhoI nuclease gene (ML13270) displayed DNA degradation activity at 75° C. but not at 25° C. ML13270 also displayed DNA degradation activity at 70° C. and 80° C., i.e., ±5° C. of 75° C., the optimal temperature for PhoI nuclease activity. The results show that in a Y. lipolytica host strain transformed with an exogenous nuclease gene, the genomic DNA of the host strain can be degraded within a temperature range of at least 10 degrees centered around the optimal temperature for the nuclease gene. This result was observed in two different nucleases where each nuclease has a different optimal temperature.

### Example 7

### Production of Plasmids for Bacterial Expression

A plasmid was generated for expression of TaqI thermophilic nuclease in E. coli as shown in Table 3. A codon optimized taqI nuclease ORF sequence was synthesized de novo based on the sequence of the taqI nuclease gene of Thermus aquaticus (SEQ ID NO:2), using the E. coli codon bias as specified in SEQ ID NO:11. This codon-optimized taqI nuclease ORF was cleaved using NcoI and Acc65I and ligated to pMB4124 (Kan^{R} oriP15) cut with NcoI and Acc65I to produce pMB7268 (trcP-taqI). The resulting encoded TaqI protein of pMB7268 is specified in SEQ ID NO:3.

For expression of TaqI thermophilic nuclease in Corynebacterium glutamicum, a fragment was generated by PCR with oligos MO9315 and MO9316 and pMB6736 as template, cleaved with NcoI and KpnI, and ligated to pMB4124 cut with NcoI and Acc65I to produce pMB7263 (trcP-taqI). The resulting encoded TaqI protein of pMB7263 is specified in SEQ ID NO:3. The sequence of primer MO9315 is specified in SEQ ID NO:12. The sequence of primer MO9316 is specified in SEQ ID NO:13.

TABLE 3
Plasmids

| Plasmid | Backbone | Insert | Oligos or source |
|---|---|---|---|
| pMB4124 | KanR oriP15 lacI^{q} trcP | lacZ | Previously |
| | | | synthesized |
| pMB7268 | pMB4124 | taqI | Synthesized |
| | | | NcoI-Acc65I |
| | | | fragment |
| pMB7263 | pMB4124 | taqI | PCR amplified |
| | | | using oligos |
| | | | MO9315 and |
| | | | MO9316 and |
| | | | pMB6736 as |
| | | | template |

### Example 8

### Demonstration of Nuclease Activity of E. coli Expressing TaqI Nuclease

In this example, the gene encoding TaqI nuclease was introduced into a host strain of E. coli. The TaqI nuclease activity in the host strain was tested.

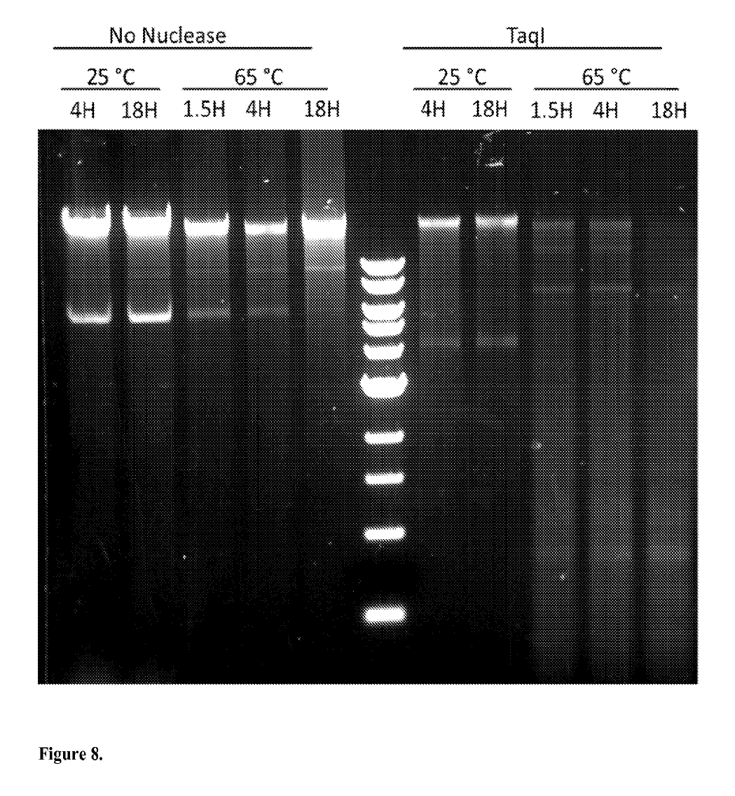

E. coli DH5a (F-Φ80lacZΔM15 Δ(lacZYA-argF) U169 recA1 endA1 hsdR17 (rK−, mK+) phoA supE44 λ-thi-1 gyrA96 relA1) harboring either pMB4124 (trcP-lacZ) or pMB7268 (trcP-taqI) was grown to exponential phase at 30° C., shifted to 37° C. for 1 hr, and induced with 500 μM isopropyl β-D-1-thiogalactopyranoside (IPTG) for 2.5 hr. Cells were then incubated at 25° C. for 4 and 18 hr, or at 65° C. for 1.5, 4, and 18 hr. Total DNA was prepared from ˜1 A₆₀₀ unit of cells using a Qiagen Blood and Tissue DNeasy Kit, following the manufacturer's recommendations, except that the incubation temperature for the Proteinase K digestion was changed to 22° C. As seen in FIG. 8, the cells harboring the trcP-taqI construct showed extensive DNA degradation at 65° C. but not at 25° C. The control cells lacking the trcP-taqI construct do not exhibit degradation at either temperature.

### Example 9

### Demonstration of Nuclease Activity of C. glutamicum Expressing TaqI Nuclease

In this example, the gene encoding TaqI nuclease was introduced into a host strain of C. glutamicum. The TaqI nuclease activity in the host strain was tested.

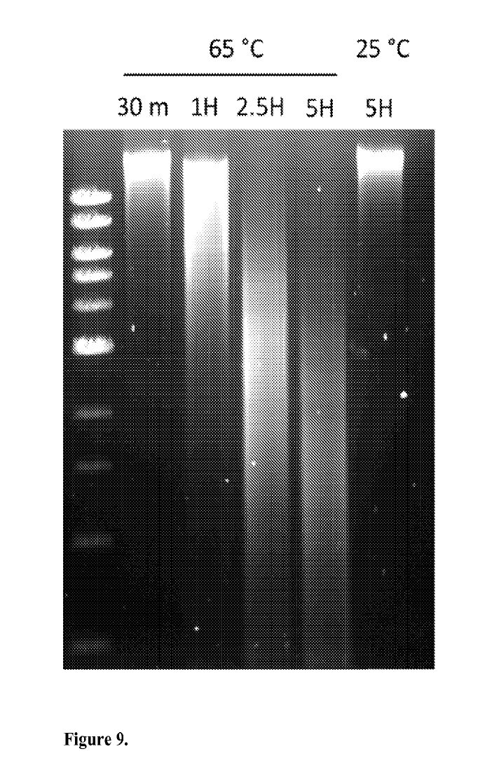

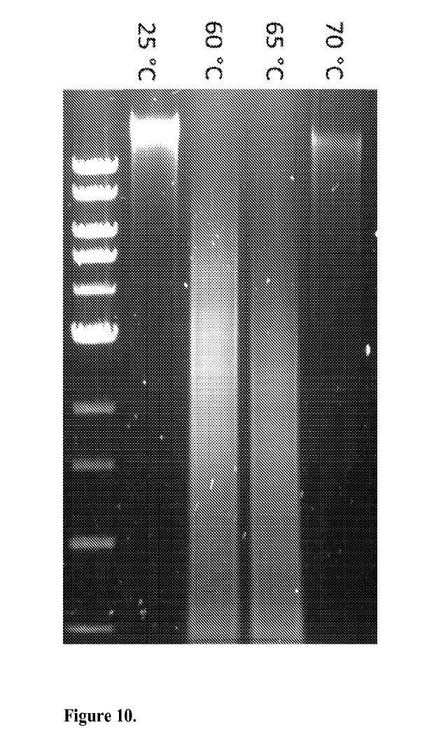

C. glutamicum strain MA428 was constructed by replacing the native hom(thrA)-thrB operon in ATCC 13032 with a feedback-resistant allele (G362E) of Streptomyces coelicolor hom driven by the C. glutamicum gpd promoter (U.S. patent application Ser. No. 10/858,730, now abandoned). This strain was transformed with pMB7263 (trcP-taqI), and a Kan^{R} colony was grown at 30° C. in BHI medium supplemented with 20 μg/ml kanamycin, and induced overnight with 250 μM IPTG. Cells were then incubated at 25, 60, 65 or 70° C., and DNA was extracted using a Qiagen Blood and Tissue DNeasy Kit, following the manufacturer's recommendations for gram-positive bacteria, except that the incubation temperature for the Proteinase K digestion was changed to 22° C. Cells harboring the trcP-taqI construct were incubated at 25° C. for 5 hrs and at 65° C. for 0.5, 1, 2.5 and 5 hrs. As seen in FIG. 9, cells containing the trcP-taqI construct showed extensive DNA degradation at 65° C. but not at 25° C. FIG. 10 depicts the temperature dependence of the activity (all incubations were for 5 hrs). The genomic DNA of the host strain C. glutamicum was degraded within a temperature range of at least 10 degrees centered around the optimal temperature for TaqI nuclease of 65° C.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

What is claimed is:

1. A genetically modified cell which comprises a heterologous nucleic acid sequence encoding a thermophilic nuclease gene, wherein the thermophilic nuclease produced by said thermophilic nuclease gene is latent at the temperature at which the cell has normal growth, wherein the genetically modified cell produces one or more end products, and wherein said one or more end products are not the thermophilic nuclease.

2. The cell of claim 1, wherein the nuclease is a DNA-degrading nuclease.

3. The cell of claim 1, wherein the nuclease is a RNA-degrading nuclease.

4. The cell of claim 2, wherein the DNA-degrading nuclease is TaqI nuclease.

5. The cell of claim 2, wherein the DNA-degrading nuclease is PhoI nuclease.

6. The cell of any of claims 1-5, wherein the genetically modified cell is from a plant.

7. The cell of any of claims 1-5, wherein the genetically modified cell is from an animal.

8. The cell of any of claims 1-5, wherein the genetically modified cell is from a microorganism.

9. The cell of claim 8, wherein the microorganism is selected from a group consisting of: yeast, fungi, algae, bacteria, and archaea.

10. The cell of claim 9, wherein the microorganism is selected from a group consisting of: Yarrowia, Bacillus, Escherichia, Pseudomonas, Paracoccus, Corynebacterium, Candida, Hansenula, Saccharomyces, Mortierella, Schizosaccharomyces, Aspergillus, Fusarium, Trichoderma, Crypthecodinium, Schizochytrium, and Thraustochytrium.

11. The cell of claim 10, wherein the Yarrowia species is Yarrowia lipolytica.

12. The cell of claim 11, wherein said end product is selected from the group consisting of: phytoene, lycopene, beta-carotene, alpha-carotene, beta-cryptoxanthin, lutein, zeaxanthin, astaxanthin, canthaxanthin, echinenone, 3-hydroxyechinenone, 3′-hydroxyechinenone, adonirubin, violaxanthin, adonixanthin, ubiquinone, vitamin K, vitamin E, retinol, retinal, retinoic acid, retinyl palmitate, and modified forms thereof.

13. The cell of claim 1, wherein the gene of the thermophilic nuclease is codon optimized to match the codon usage bias of the host cell.

14. The cell of claim 4, wherein the TaqI nuclease comprises a polypeptide having an amino acid sequence of SEQ ID NO:3.

15. The cell of claim 5, wherein the PhoI nuclease comprises a polypeptide having an amino acid sequence of SEQ ID NO:10.

16. The cell of claim 1, wherein the production of said end products has little or no change comparing to the production of said end products from a cell which does not contain said heterologous nucleic acid sequence.

17. The cell of claim 1, wherein the cell comprises a combination of more than one thermophilic nuclease gene.

18. A method for degrading the nucleic acids of a host cell in vivo and/or in situ, wherein the host cell comprises a heterologous nucleic acid sequence encoding a thermophilic nuclease gene, wherein the method comprises:
a) growing the host cell at a temperature at which the thermophilic nuclease is latent; and
b) degrading the nucleic acids of the host cell by changing the temperature in step (a) to a temperature at which the thermophilic nuclease is active.

19. The method of claim 18, wherein the nuclease is a DNA-degrading nuclease and the nucleic acids are DNA.

20. The method of claim 19, wherein the DNA of the host cell contains recombinant DNA.

21. The method of claim 18, wherein the nuclease is a RNA-degrading nuclease and the nucleic acids are RNA.

22. The method of any of claims 18-21, wherein the temperature in step (a) is optimal for the growth of the host cell.

23. The method of any of claims 18-21, wherein the degradation is conducted at a temperature that is within about ±5° C. of the optimum temperature of said thermophilic nuclease.

24. The method of claim 23, wherein the degradation is conducted at a temperature that is optimal for the activity of the thermophilic nuclease.

25. The method of claim 18, wherein the degradation is conducted during the pasteurization of the host cell.

26. The method of claim 18, wherein the degradation is conducted in a temperature range between 40° C. and 100° C.

27. The method of claim 19, wherein the DNA-degrading nuclease is TaqI nuclease.

28. The method of claim 27, wherein the degradation is conducted at a temperature ranging between about 60° C. and about 70° C.

29. The method of claim 27, wherein the degradation is conducted at 65° C.

30. The method of claim 19, wherein the DNA-degrading nuclease is PhoI nuclease.

31. The method of claim 30, wherein the degradation is conducted at a temperature ranging between about 70° C. and about 80° C.

32. The method of claim 31, wherein the degradation is conducted at 75° C.

33. The method of any of claims 18-32, wherein the host cell is from a plant.

34. The method of any of claims 18-32, wherein the host cell is from an animal.

35. The method of any of claims 18-32, wherein the host cell is from a microorganism.

36. The method of claim 35, wherein the microorganism is selected from a group consisting of: yeast, fungi, algae, bacteria, and archaea.

37. The method of claim 36, wherein the microorganism is selected from a group consisting of: Yarrowia, Bacillus, Escherichia, Pseudomonas, Paracoccus, Corynebacterium, Candida, Hansenula, Saccharomyces, Mortierella, Schizosaccharomyces, Aspergillus, Fusarium, Trichoderma, Crypthecodinium, Schizochytrium, and Thraustochytrium.

38. The method of claim 37, wherein the Yarrowia species is Yarrowia lipolytica.

39. The method of claim 18, wherein the gene of the thermophilic nuclease is codon optimized to match the codon usage bias of the host cell.

40. The method of claim 27, wherein the TaqI nuclease comprises a polypeptide having an amino acid sequence of SEQ ID NO:3.

41. The method of claim 30, wherein the PhoI nuclease comprises a polypeptide having an amino acid sequence of SEQ ID NO:10.

42. The method of claim 18, wherein more than one thermophilic nuclease gene is introduced into the host cell.

43. The method of claim 20, wherein said recombinant DNA is optimized to include additional sites of the thermophilic nuclease.

44. The method of claim 20, wherein said recombinant DNA is optimized to include one cleavage site every 500 or fewer nucleotides.

45. Use of a thermophilic nuclease for degrading the nucleic acids of a host cell in vivo and/or in situ, wherein the thermophilic nuclease is heterologous to the host cell.

46. The use of claim 45, wherein the nuclease is a DNA-degrading nuclease and the nucleic acids are DNA.

47. The use of claim 46, wherein the DNA of the host cell contains recombinant DNA.

48. (canceled)

49. The use of any of claims 45-48, wherein the degradation is conducted at a temperature that is within about ±5° C. of the optimum temperature of said thermophilic nuclease.

50-70. (canceled)

71. A process for the production of a biomass product which is free of active nucleic acid molecules, wherein the biomass product is the product of a host cell comprising a heterologous nucleic acid sequence encoding a thermophilic nuclease gene, wherein the process comprises:
a) fermenting the host cell at a temperature at which the thermophilic nuclease is latent;
b) degrading the nucleic acids of the cell by changing the temperature in step (a) to a temperature at which the thermophilic nuclease is active; and
c) recovering the biomass product;
wherein the order for performing steps b) and step c) may be exchanged.

72. The process of claim 71, wherein the nuclease is a DNA-degrading nuclease and the nucleic acids are DNA.

73. The process of claim 72, wherein the DNA of the host cell contains recombinant DNA.

74. (canceled)

75. The process of any of claims 71-74, wherein the temperature in step (a) is optimal for the growth of the host cell.

76. The process of any of claims 71-74, wherein the degradation is conducted at a temperature that is within about ±5° C. of the optimum temperature of said thermophilic nuclease.

77-97. (canceled)
